(19) United States
(12) Patent Application Publication
(10) Pub. No.: US 2008/0280877 A1
(43) Pub. Date: Nov. 13, 2008

(54) Azetidines

(75) Inventors: Kevin Neil Dack, Sandwich (GB); Sarah Elizabeth Skerratt, Sandwich (GB); Siew Kuen Yeap, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 12/115,070

(22) Filed: May 5, 2008

Related U.S. Application Data
(60) Provisional application No. 61/013,030, filed on Dec. 12, 2007.
(60) Provisional application No. 60/917,081, filed on May 10, 2007.

(51) Int. Cl. A61K 31/497; C07D 205/04; A61K 31/397; A61P 15/00; C07D 403/12

(52) U.S. Cl. 514/210.18; 548/953; 514/210.17; 544/408

(57) ABSTRACT

The invention relates to EP2 antagonist azetidines of formula (I)

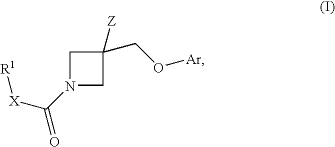

wherein Ar, R¹, X, and Z are as defined herein, to their use in medicine, particularly in the treatment of endometriosis and/or uterine fibroids, to intermediates useful in their synthesis, and to compositions containing them.

This application claims priority to U.S. Provisional Application Ser. Nos. 61/013,030 and 60/917,081, filed Dec. 12, 2007 and May 10, 2007, respectively.

This present invention relates to azetidine compounds, their pharmaceutically acceptable salts, solvates, hydrates, and prodrugs thereof, to their use in medicine, to compositions containing them, to processes for their preparation, and to intermediates used in such processes. The compounds are preferably antagonists at the prostaglandin E₂ (PGE₂) receptor-2 (also known as the EP2 receptor). More preferably the compounds are EP2 antagonists with selectivity over DP1 (prostaglandin D1 receptor) and/or EP4 (prostaglandin E₄ (PGE₄) receptor-4). Most preferably the compounds are EP2 antagonists with selectivity over DP1 and EP4. In particular the present invention relates to a class of azetidine compounds which should be useful for the treatment of EP2-mediated conditions, such as endometriosis, uterine fibroids (leiomyomata), menorrhagia, adenomyosis, primary and secondary dysmenorrhoea (including symptoms of dyspareunia, dyschexia and chronic pelvic pain), chronic pelvic pain syndrome.

Endometriosis is a common gynaecological disease that affects 10-20% women of reproductive age and manifests itself in the presence of functional ectopic endometrial glands and stroma at locations outside the uterine cavity (reviewed in (Prentice 2001)). Patients with endometriosis may present with many different symptoms and severity. Most commonly this is dysmenorrhoea, but chronic pelvic pain, dyspareunia, dyschezia, menorrhagia, lower abdominal or back pain, infertility, bloating and pain on micturition are also part of the constellation of symptoms of endometriosis.

Originally described by Von Rokitansky in 1860 (Von Rokitansky 1860), the exact pathogenesis of endometriosis is unclear (Witz 1999; Witz 2002), but the most widely accepted theory is the implantation, or Sampson, theory (Sampson 1927). The Sampson theory postulates that the development of endometriosis is a consequence of retrograde dissemination and implantation of endometrial tissue into the peritoneal cavity during menstruation. Following attachment, the fragments of endometrium recruit a vascular supply and undergo cycles of proliferation and shedding under local and systemic hormonal controls. In women with patent fallopian tubes, retrograde menstruation appears to be a universal phenomenon (Liu & Hitchcock 1986). The disease often manifests itself as rectovaginal endometriosis or adenomyosis, ovarian cystic endometriomas and, most commonly, peritoneal endometriosis. The major sites of attachment and lesion growth within the pelvis are the ovaries, broad and round ligaments, fallopian tubes, cervix, vagina, peritoneum and the pouch of Douglas. At its most severe, endometriosis can cause profound structural modification to peritoneal cavity, including multiorgan adhesions and fibrosis.

Symptomatic endometriosis can be managed medically and surgically, where the intention is to remove the ectopic lesion tissue. Surgical intervention can be either conservative, aiming to preserve the reproductive potential of the patient, or comparatively radical for severe disease, involving dissection of the urinary tract, bowel, and rectovaginal septum, or total abdominal hysterectomy and bilateral salpingo-oopherectomy. Medical pharmacological treatments such as the androgenic therapies, danazol and gestrinone, the constellation of GnRH agonists, buserelin, goserelin, leuprolide, nafarelin and triptorelin, GnRH antagonists, cetrorelix and abarelix, as well as the progestogens, including medroxyprogesterone acetate, induce lesion atrophy by suppressing the production of estrogen. These approaches are not without unwanted side effects; danazol and gestrinone include weight gain, hirsuitism, acne, mood changes and metabolic effects on the cardiovascular system. The group of GnRH agonists and antagonists are found to cause a profound suppression of estrogen leading to vasomotor effects (hot flashes) and depletion of bone mineral density, which restricts their use to only six months of therapy.

Uterine leiomyomas (Walker 2002; Flake, et al. 2003), or fibroids, are the most common benign tumours found in women and occur in the majority of women by the time they reach the menopause. Although uterine fibroids are the most frequent indication for hysterectomy in the United States, as with endometriosis, remarkably little is known about the underlying pathophysiology of the disease. As with endometriotic lesions, the presence of enlarged uterine fibroids is associated with abnormal uterine bleeding, dysmenorrhoea, pelvic pain and infertility. Outside of surgical management, medical treatments commonly used for endometriosis, such as GnRH analogues or danazol, have been shown to suppress fibroid growth by inducing a reversible hypoestrogenic state (Chrisp & Goa 1990; Chrisp & Goa 1991; De Leo, et al. 2002; Ishihara, et al. 2003).

However, the future disease management of both uterine fibroids and endometriosis will rely on the development of more effective, well-tolerated and safer agents than those that are currently available. There are long term deleterious effects (altered sexual function, decreases in bone mineral density as well as increased risk of cardiovascular and thrombotic complications) of existing agents that completely suppress ovarian function and lead to decreases in bone mineral density, there is a motivation for developing non-hormonal mechanisms or approaches which modify the disease specifically at the level of the ectopic disease. One of these includes approaches includes agents which modify the cyclooxygenase-2 (COX-2) dependent PGE₂ signalling pathway (Boice & Rohrer 2005). PGE₂ mediates its effects through G protein-coupled receptors EP1, EP2, EP3 and EP4. Both the differential expression of EP receptors as well as their intracellular coupling pathways mediates the diverse biological functions of PGE₂ in different cell types (Narumiya, et al. 1999; Tilley, et al. 2001). The EP2 and EP4 receptors specifically couple to G proteins which activate adenylate cyclase and lead to the production of cAMP. In the uterine endometrium, COX-2 expression increases on glandular epithelium in the proliferative phase and is accompanied by an increase in EP2 and EP4 receptor expression (reviewed by (Sales & Jabbour 2003; Jabbour, et al. 2006)). In pathological conditions of the endometrium, such as endometrial adenocarcinoma, adenomyosis and endometriosis, this pathway appears to be up regulated (Jabbour, et al. 2001; Ota, et al. 2001; Chishima, et al. 2002; Jabbour 2003; Matsuzaki, et al. 2004b; Buchweitz, et al. 2006). COX-2 plays an important role in ovulation, implantation, decidualisation and parturition (Sales & Jabbour 2003). Mice in which the EP2 receptor is deleted by homologous recombination have defects in embryo implantation and fertility (Hizaki, et al. 1999; Kennedy, et al. 1999; Tilley, et al. 1999), supporting the notion that COX-2 derived PGE₂ mediates effects on the uterine endometrium in part through the EP2 receptor. The expression of COX-2 is known to be greatly up regulated at ectopic sites of disease, in contrast to that on normal eutopic endometrium (Ota, et al. 2001; Chishima, et al. 2002; Matsuzaki, et al. 2004b; Buchweitz, et al. 2006) and PGE2 induces the proliferation of endometrial epithelial cells in culture (Jabbour & Boddy 2003). In pre-clinical disease models of endometriosis, treatment with COX-2 selective agents, leads to the decrease in disease burden (Dogan, et al. 2004; Matsuzaki, et al. 2004a; Ozawa, et al. 2006; Laschke, et al. 2007). There is also one published clinical study (Cobellis, et al. 2004) which indicates that treatment of patients with endometriosis with rofecoxib for 6 months leads to improvements in pain symptoms and outcomes compared with placebo.

The aberrant expression of COX-2 in patients with endometriosis appears to have a number of consequences (Sales & Jabbour 2003). First, PGE₂ appears to augment the expression and activity of aromatase on ectopic endometrial stromal cells (Noble, et al. 1997; Zeitoun & Bulun 1999). It could be speculated that ectopic generation of aromatase by the lesion would lead to increased local estrogen production, driving lesion growth independently of ovarian control and the normal estrous cycle. That the effects of PGE₂ on aromatase expression in vitro can be mimicked by the selective EP2 receptor agonist, butaprost (Zeitoun & Bulun 1999), supports the notion that compounds of the present invention would have utility in the treatment of growth disorders which are driven ectopic aromatase expression, such as endometriosis, adenomyomas, uterine fibroids as well as uterine and breast carcinoma.

There are other possible mechanisms by which a selective EP2 antagonist might inhibit cell growth. The observed effects of COX-2 inhibitors, such as celecoxib, in preventing intestinal polyp formation (Arber, et al. 2006) and the protection from adenoma formation in a mouse model ( [symbol] APC mouse) of familial adenomatous polyposis complex by deletion of COX-2 (Oshima, et al. 1996; Oshima, et al. 2001), implies that the PGE₂ pathway also has a key role in promoting carcinoma growth. That polyp and adenoma formation in the [symbol] APC mouse model can also be inhibited by crossing these by additional germline deletion of the EP2 receptor, is consistent with the view that PGE₂ mediates effects on cell differentiation and growth through the EP2 receptor (Sonoshita, et al. 2001; Seno, et al. 2002). Furthermore, the emerging knowledge of the downstream signalling pathway from the EP2 receptor is consistent with EP2 playing a key role in early G1 events in cell cycle control, such as the regulation of [symbol] -catenin (Castellone, et al. 2005; Castellone, et al. 2006) and MAP kinase pathways (Jabbour & Boddy 2003).

Angiogenesis, the sprouting of capillaries from pre-existing vasculature, occurs during embryo development, wound repair and tumour growth. The increased COX-2 expression and vascular densities which accompany the development of adenomas in the [symbol] APC mouse, are also consistently observed in clinical specimens and pre-clinical models of endometriosis and malignant conditions of, including but not limited to, ovarian, dermal, prostate, gastric, colorectal and breast cancer (Subbaramaiah, et al. 2002; Hull, et al. 2003; Kamiyama, et al. 2006). The involvement of the COX-2 pathway in this process has been supported by a number of observations (Liu, et al. 2001; Leahy, et al. 2002; Chang, et al. 2004; Ozawa, et al. 2006). The peritoneal fluid of women with endometriosis appears to display greater angiogenic activity than women without endometriosis (Gazvani & Templeton 2002; Bourlev, et al. 2006) and PGE₂ has been shown to promote the transcription of angiogenic factors such as VEGF and angiopoietins (reviewed in (Gately & Li 2004)). Recent data that indicate the specific contribution of EP2 receptors in the stimulation of endothelial cell growth and migration (Kamiyama, et al. 2006) as well as response to hypoxia (Critchley, et al. 2006), is consistent with and supports the notion that compounds of the present invention would have utility in the treatment of angiogenic disorders including, but not limited to, endometriosis, adenomyosis, leiomyoma, menorrhagia, macular degeneration, rheumatoid arthritis and cancer.

Both uterine nerve ablation and pre-sacral neurectomy surgical techniques are used to manage the painful symptoms of primary and secondary dysmenorrhoea (Proctor, et al. 2005). As PGE₂ is generated from PGH₂ by the action of COX-1 and COX-2 on arachadonic acid, elevated PGE₂ would have direct, pain-sensitizing effects on sensory afferent fibres that innervate the peritoneum and ectopic lesions (Tulandi, et al. 2001; Al-Fozan, et al. 2004; Berkley, et al. 2004; Quinn & Armstrong 2004; Tokushige, et al. 2006a; Tokushige, et al. 2006b). That elevated COX-2 expression correlates with non-menstrual chronic pelvic pain (Buchweitz, et al. 2006) is consistent with this notion. A number of lines of evidence from studies in mouse models suggest that one of the modes of action of PGE₂ on pain and nociception is mediated by the EP2 receptor (Ahmadi, et al. 2002; Reinold, et al. 2005; Hosl, et al. 2006). As such compounds of the present invention would have utility in the treatment of pain disorders including, but not limited to, dysmenorrhoea, dyschezia, dyspareunia, irritable bowel syndrome, endometriosis, adenomyosis, leiomyomata, CPP, interstitial cystitis, inflammatory and neuropathic pain conditions.

During the development of endometriosis activated inflammatory cells appear to be recruited into the peritoneal cavity. Peritoneal macrophages from women with endometriosis release more PGE₂ than those without endometriosis (Karck, et al. 1996; Wu, et al. 2005). One of the effects of elevated levels PGE₂ on peritoneal macrophages is to inhibit MMP-9 expression and thereby attenuate macrophage phagocytic function (Wu, et al. 2005), leading to the prolonged accumulation of endometrial tissue in the peritoneum. As such by restoring macrophage function, these findings give further support to the use of compounds of the present invention in the treatment of endometriosis and cancer.

Known EP2 antagonists include AH6809, (Pelletier, et al. 2001), but both its potency and selectivity fall short of being suitable for medical therapy.

S. Ahmadi, S. Lippross, W. L. Neuhuber & H. U. Zeilhofer. PGE2 selectively blocks inhibitory glycinergic neurotransmission onto rat superficial dorsal horn neurons. Nat Neurosci 5, 34-40 (2002).

H. Al-Fozan, S. Bakare, M.-F. Chen & T. Tulandi. Nerve fibers in ovarian dermoid cysts and endometriomas. Fertil Steril 82, 230-1 (2004).

N. Arber, C. J. Eagle, J. Spicak, I. Racz, P. Dite, J. Hajer, M. Zavoral, M. J. Lechuga, P. Gerletti, J. Tang, R. B. Rosenstein, K. Macdonald, P. Bhadra, R. Fowler, J. Wittes, A. G. Zauber, S. D. Solomon & B. Levin. Celecoxib for the prevention of colorectal adenomatous polyps. N Engl J Med. 355, 885-95. (2006).

K. J. Berkley, N. Dmitrieva, K. S. Curtis & R. E. Papka. Innervation of ectopic endometrium in a rat model of endometriosis. PNAS 101, 11094-8 (2004).

J. A. Boice & S. Rohrer 2005 Use of selective cyclooxygenase-2 inhibitors for the treatment of endometriosis 2004-US19441 2005000238

V. Bourlev, N. Volkov, S. Pavlovitch, N. Lets, A. Larsson & M. Olovsson. The relationship between microvessel density, proliferative activity and expression of vascular endothelial growth factor-A and its receptors in eutopic endometrium and endometriotic lesions. Reproduction 132, 501-09 (2006).

O. Buchweitz, A. Staebler, P. Wuelfing, E. Hauzman, R. Greb & L. Kiesel. COX-2 overexpression in peritoneal lesions is correlated with non-menstrual chronic pelvic pain. European Journal of Obstetrics & Gynecology and Reproductive Biology 124, 216-21 (2006).

M. D. Castellone, H. Teramoto, B. O. Williams, K. M. Druey & J. S. Gutkind. Prostaglandin E2 promotes colon cancer cell growth Through a Gs-axin-b-catenin signaling axis. Science (Washington, D.C., United States) 310, 1504-10 (2005).

M. D. Castellone, H. Teramoto & J. S. Gutkind. Cyclooxygenase-2 and colorectal cancer chemoprevention: The beta-catenin connection. Cancer Research 66, 11085-88 (2006).

S.-H. Chang, C. H. Liu, R. Conway, D. K. Han, K. Nithipatikom, O. C. Trifan, T. F. Lane & T. Hla. From the Cover: Role of prostaglandin E2-dependent angiogenic switch in cyclooxygenase 2-induced breast cancer progression. PNAS 101, 591-96 (2004).

F. Chishima, S. Hayakawa, K. Sugita, N. Kinukawa, S. Aleemuzzaman, N. Nemoto, T. Yamamoto & M. Honda. Increased expression of cyclooxygenase-2 in local lesions of endometriosis patients. Am J Reprod Immunol 48, 50-6 (2002).

P. Chrisp & K. L. Goa. Nafarelin. A review of its pharmacodynamic and pharmacokinetic properties, and clinical potential in sex hormone-related conditions. Review 74 refs. Drugs 39, 523-51 (1990).

P. Chrisp & K. L. Goa. Goserelin. A review of its pharmacodynamic and pharmacokinetic properties, and clinical use in sex hormone-related conditions. Review 155 refs. Drugs 41, 254-88 (1991).

L. Cobellis, S. Razzi, S. De Simone, A. Sartini, A. Fava, S. Danero, W. Gioffre, M. Mazzini & F. Petraglia. The treatment with a COX-2 specific inhibitor is effective in the management of pain related to endometriosis. European Journal of Obstetrics & Gynecology and Reproductive Biology 116, 100-02 (2004).

H. O. D. Critchley, J. Osei, T. A. Henderson, L. Boswell, K. J. Sales, H. N. Jabbour & N. Hirani. Hypoxia-Inducible Factor-1{alpha} Expression in Human Endometrium and Its Regulation by Prostaglandin E-Series Prostanoid Receptor 2 (EP2). Endocrinology 147, 744-53 (2006).

V. De Leo, G. Morgante, A. La Marca, M. C. Musacchio, M. Sorace, C. Cavicchioli & F. Petraglia. A benefit-risk assessment of medical treatment for uterine leiomyomas. Drug Safety 25, 759-79 (2002).

E. Dogan, U. Saygili, C. Posaci, B. Tuna, S. Caliskan, S. Altunyurt & B. Saatli. Regression of endometrial explants in rats treated with the cyclooxygenase-2 inhibitor rofecoxib. Fertil Steril. 82 Suppl 3, 1115-20. (2004).

G. P. Flake, J. Andersen & D. Dixon. Etiology and pathogenesis of uterine leiomyomas: a review. Environmental Health Perspectives 111, 1037-54 (2003).

S. Gately & W. W. Li. Multiple roles of COX-2 in tumor angiogenesis: a target for antiangiogenic therapy. Seminars in Oncology 31, 2-11 (2004).

R. Gazvani & A. Templeton. Peritoneal environment, cytokines and angiogenesis in the pathophysiology of endometriosis. Reproduction 123, 217-26 (2002).

H. Hizaki, E. Segi, Y. Sugimoto, M. Hirose, T. Saji, F. Ushikubi, T. Matsuoka, Y. Noda, T. Tanaka, N. Yoshida, S. Narumiya & A. Ichikawa. Abortive expansion of the cumulus and impaired fertility in mice lacking the prostaglandin E receptor subtype EP2. PNAS 96, 10501-06 (1999).

K. Hosl, H. Reinold, R. J. Harvey, U. Muller, S. Narumiya & H. U. Zeilhofer. Spinal prostaglandin E receptors of the EP2 subtype and the glycine receptor [alpha]3 subunit, which mediate central inflammatory hyperalgesia, do not contribute to pain after peripheral nerve injury or formalin injection. Pain 126, 46-53 (2006).

M. L. Hull, D. S. Charnock-Jones, C. L. K. Chan, K. L. Bruner-Tran, K. G. Osteen, B. D. M. Tom, T.-P. D. Fan & S. K. Smith. Antiangiogenic Agents Are Effective Inhibitors of Endometriosis. J Clin Endocrinol Metab 88, 2889-99 (2003).

H. Ishihara, J. Kitawaki, N. Kado, H. Koshiba, S. Fushiki & H. Honjo. Gonadotropin-releasing hormone agonist and danazol normalize aromatase cytochrome P450 expression in eutopic endometrium from women with endometriosis, adenomyosis, or leiomyomas. Fertility & Sterility 79, 735-42 (2003).

H. N. Jabbour, S. A. Milne, A. R. Williams, R. A. Anderson & S. C. Boddy. Expression of COX-2 and PGE synthase and synthesis of PGE(2)in endometrial adenocarcinoma: a possible autocrine/paracrine regulation of neoplastic cell function via EP2/EP4 receptors. Br J Cancer. 85, 1023-31. (2001).

H. N. Jabbour 2003 Use of prostaglandin E synthase inhibitors, or EP2 or EP4 receptor antagonists, in the treatment of a pathological condition of the uterus 2002-GB4549 2003030911

H. N. Jabbour & S. C. Boddy. Prostaglandin E2 Induces Proliferation of Glandular Epithelial Cells of the Human Endometrium via Extracellular Regulated Kinase ½-Mediated Pathway. J Clin Endocrinol Metab 88, 4481-87 (2003).

H. N. Jabbour, K. J. Sales, O. P. Smith, S. Battersby & S. C. Boddy. Prostaglandin receptors are mediators of vascular function in endometrial pathologies. Mol Cell Endocrinol. 252, 191-200 (2006).

M. Kamiyama, A. Pozzi, L. Yang, L. M. DeBusk, R. M. Breyer & P. C. Lin. EP2, a receptor for PGE2, regulates tumor angiogenesis through direct effects on endothelial cell motility and survival. Oncogene 25, 7019-28 (2006).

U. Karck, F. Reister, W. Schafer, H. P. Zahradnik & M. Breckwoldt. PGE2 and PGF2 alpha release by human peritoneal macrophages in endometriosis. Prostaglandins. 51, 49-60. (1996).

C. R. J. Kennedy, Y. Zhang, S. Brandon, Y. Guan, K. Coffee, C. D. Funk, M. A. Magnuson, J. A. Oates, M. D. Breyer & R. M. Breyer. Salt-sensitive hypertension and reduced fertility in mice lacking the prostaglandin EP2 receptor. Nat Med 5, 217-20 (1999).

M. W. Laschke, A. Elitzsch, C. Scheuer, B. Vollmar & M. D. Menger. Selective cyclo-oxygenase-2 inhibition induces regression of autologous endometrial grafts by down-regulation of vascular endothelial growth factor-mediated angiogenesis and stimulation of caspase-3-dependent apoptosis. Fertility and Sterility 87, 163-71 (2007).

K. M. Leahy, R. L. Ornberg, Y. Wang, B. S. Zweifel, A. T. Koki & J. L. Masferrer. Cyclooxygenase-2 Inhibition by Celecoxib Reduces Proliferation and Induces Apoptosis in Angiogenic Endothelial Cells in Vivo. Cancer Res 62, 625-31 (2002).

C. H. Liu, S.-H. Chang, K. Narko, O. C. Trifan, M.-T. Wu, E. Smith, C. Haudenschild, T. F. Lane & T. Hla. Overexpression of Cyclooxygenase-2 Is Sufficient to Induce Tumorigenesis in Transgenic Mice. J. Biol. Chem. 276, 18563-69 (2001).

D. T. Liu & A. Hitchcock. Endometriosis:its association with retrograde menstruation, dysmenorrhoea and tubal pathology. British Journal of Obstetrics & Gynaecology 93, 859-62 (1986).

S. Matsuzaki, M. Canis, C. Darcha, R. Dallel, K. Okamura & G. Mage. Cyclooxygenase-2 selective inhibitor prevents implantation of eutopic endometrium to ectopic sites in rats. Fertility and Sterility 82, 1609-15 (2004a).

S. Matsuzaki, M. Canis, J.-L. Pouly, A. Wattiez, K. Okamura & G. Mage. Cyclooxygenase-2 expression in deep endometriosis and matched eutopic endometrium. Fertility and Sterility 82, 1309-15 (2004b).

S. Narumiya, Y. Sugimoto & F. Ushikubi. Prostanoid receptors: structures, properties, and functions. Physiol Rev. 79, 1193-226. (1999).

L. S. Noble, K. Takayama, K. M. Zeitoun, J. M. Putman, D. A. Johns, M. M. Hinshelwood, V. R. Agarwal, Y. Zhao, B. R. Carr & S. E. Bulun. Prostaglandin E2 stimulates aromatase expression in endometriosis-derived stromal cells. Journal of Clinical Endocrinology and Metabolism 82, 600-06 (1997).

M. Oshima, J. E. Dinchuk, S. L. Kargman, H. Oshima, B. Hancock, E. Kwong, J. M. Trzaskos, J. F. Evans & M. M. Taketo. Suppression of intestinal polyposis in Apc D716 knockout mice by inhibition of cyclooxygenase 2 (COX-2). Cell 87, 803-9 (1996).

M. Oshima, N. Murai, S. Kargman, M. Arguello, P. Luk, E. Kwong, M. M. Taketo & J. F. Evans. Chemoprevention of intestinal polyposis in the ApcD716 mouse by rofecoxib, a specific cyclooxygenase-2 inhibitor. Cancer Res 61, 1733-40 (2001).

H. Ota, S. Igarashi, M. Sasaki & T. Tanaka. Distribution of cyclooxygenase-2 in eutopic and ectopic endometrium in endometriosis and adenomyosis. Hum Reprod. 16, 561-6. (2001).

Y. Ozawa, T. Murakami, M. Tamura, Y. Terada, N. Yaegashi & K. Okamura. A selective cyclooxygenase-2 inhibitor suppresses the growth of endometriosis xenografts via antiangiogenic activity in severe combined immunodeficiency mice. Fertility and Sterility 86, 1146-51 (2006).

S. Pelletier, J. Dube, A. Villeneuve, F. Gobeil, Q. Yang, B. Battistini, G. Guillemette & P. Sirois. Prostaglandin E2 increases cyclic AMP and inhibits endothelin-1 production/secretion by guinea-pig tracheal epithelial cells through EP4 receptors. Br J Pharmacol 132, 999-1008 (2001).

A. Prentice. Regular review: Endometriosis. BMJ 323, 93-5 (2001).

M. L. Proctor, P. M. Latthe, C. M. Farquhar, K. S. Khan & N. P. Johnson. Surgical interruption of pelvic nerve pathways for primary and secondary dysmenorrhoea. Cochrane Database Syst Rev CD001896 (2005).

M. Quinn & G. Armstrong. 932-3 (Department of Obstetrics and Gynaecology, Hope Hospital, Manchester, UK., England: United Kingdom, 2004).

H. Reinold, S. Ahmadi, U. B. Depner, B. Layh, C. Heindl, M. Hamza, A. Pahl, K. Brune, S. Narumiya, U. Muller & H. U. Zeilhofer. Spinal inflammatory hyperalgesia is mediated by prostaglandin E receptors of the EP2 subtype. J. Clin. Invest. 115, 673-79 (2005).

K. J. Sales & H. N. Jabbour. Cyclooxygenase enzymes and prostaglandins in pathology of the endometrium. Reproduction 126, 559-67 (2003).

J. A. Sampson. Peritoneal endometriosis due to menstrual dissemination of endometrial tissue into the peritoneal cavity. American Journal of Obstetrics & Gynecology 14, 422-9 (1927).

H. Seno, M. Oshima, T.-O. Ishikawa, H. Oshima, K. Takaku, T. Chiba, S. Narumiya & M. M. Taketo. Cyclooxygenase 2- and prostaglandin E2 receptor EP2-dependent angiogenesis in ApcD716 mouse intestinal polyps. Cancer Research 62, 506-11 (2002).

M. Sonoshita, K. Takaku, N. Sasaki, Y. Sugimoto, F. Ushikubi, S. Narumiya, M. Oshima & M. M. Taketo. Acceleration of intestinal polyposis through prostaglandin receptor EP2 in ApcD716 knockout mice. Nature Medicine (New York, N.Y., United States) 7, 1048-51 (2001).

K. Subbaramaiah, L. Norton, W. Gerald & A. J. Dannenberg. Cyclooxygenase-2 Is Overexpressed in HER-2/neu-positive Breast Cancer. EVIDENCE FOR INVOLVEMENT OF AP-1 AND PEA3. J. Biol. Chem. 277, 18649-57 (2002).

S. L. Tilley, L. P. Audoly, E. H. Hicks, H.-S. Kim, P. J. Flannery, T. M. Coffman & B. H. Koller. Reproductive failure and reduced blood pressure in mice lacking the EP2 prostaglandin E2 receptor. J. Clin. Invest. 103, 1539-45 (1999).

S. L. Tilley, T. M. Coffman & B. H. Koller. Mixed messages: modulation of inflammation and immune responses by prostaglandins and thromboxanes. J Clin Invest. 108, 15-23. (2001).

N. Tokushige, R. Markham, P. Russell & I. S. Fraser. Nerve fibres in peritoneal endometriosis. Human Reproduction 21, 3001-07 (2006a).

N. Tokushige, R. Markham, P. Russell & I. S. Fraser. High density of small nerve fibres in the functional layer of the endometrium in women with endometriosis. Hum Reprod 21, 782-7 (2006b).

T. Tulandi, A. Felemban & M. F. Chen. Nerve fibers and histopathology of endometriosis-harboring peritoneum. J Am Assoc Gynecol Laparosc 8, 95-8 (2001).

C. Von Rokitansky. Ueber uterusdruesen-neubildung in uterus und ovarial sarcomen. Ztsch K K Gesellsch der Aerzte zu Wien 37, 577-81 (1860).

C. L. Walker. Role of hormonal and reproductive factors in the etiology and treatment of uterine leiomyoma. Recent Progress in Hormone Research 57, 277-94 (2002).

C. A. Witz. Current concepts in the pathogenesis of endometriosis. Clinical Obstetrics & Gynecology 42, 566-85 (1999).

C. A. Witz. Pathogenesis of endometriosis. Gynecologic & Obstetric Investigation 53, 52-62 (2002).

M. H. Wu, Y. Shoji, M. C. Wu, P. C. Chuang, C. C. Lin, M. F. Huang & S. J. Tsai. Suppression of matrix metalloproteinase-9 by prostaglandin E(2) in peritoneal macrophage is associated with severity of endometriosis. Am J Pathol. 167, 1061-9. (2005).

K. M. Zeitoun & S. E. Bulun. Aromatase: a key molecule in the pathophysiology of endometriosis and a therapeutic target. Fertility and Sterility 72, 961-69 (1999).

The compounds of the invention have potentially useful pharmaceutical properties including, but is not limited to, EP2 antagonist properties, useful in the treatment of endometriosis, uterine fibroids and menorrhagia, adenomyosis, primary and secondary dysmenorrhoea (including symptoms of dyspareunia, dyschexia and chronic pelvic pain), chronic pelvic pain syndrome, precocious puberty, cervical ripening, breast carcinoma, colon carcinoma, familial adenomatous polyposis, colorectal adenomas, endometrial carcinoma, prostate carcinoma, pulmonary carcinoma, testicular carcinoma, gastric carcinoma, macular degeneration, inflammatory and neuropathic pain conditions, cancer pain.

Particularly of interest are endometriosis, uterine fibroids, menorrhagia, adenomyosis, primary and secondary dysmenorrhoea, chronic pelvic pain syndrome. In particular, the compounds and derivatives of the present invention exhibit activity as prostaglandin E₂ (PGE₂) receptor-2 (EP2) antagonists and may be useful for treatment where EP2 receptor antagonism is indicated.

More particularly, the compounds and derivatives of the invention may be useful for treating endometriosis and/or uterine fibroids.

The terms “treating”, “treat”, or “treatment” as used herein are intended to embrace both prevention and control i.e., prophylactic, and palliative treatment of the indicated conditions.

The present invention provides for compounds of formula (I):

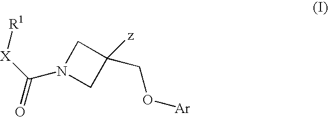

wherein
R¹ is a phenyl group (optionally substituted by one or two substituents independently selected from F, Cl, Br, CN, C₁₋₄ alkyl, C₁₋₄ alkylthio and C₁₋₄ alkoxy, per-fluoro-C₁₋₆ alkyl and perfluoro-C₁₋₆ alkoxy), or a tetrahydropyranyl group;
X represents a direct link or NH;
Z is selected from

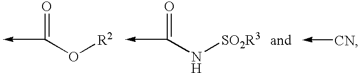

R² and R³ are H or C₁₋₆ alkyl (optionally substituted by 1 to 3 fluorine atoms);
Ar is an aromatic group consisting of 1, 2 or 3 aromatic rings, which aromatic rings are independently selected from phenyl and a 5- or 6-membered heteroaromatic ring containing 1, 2 or 3 heteroatoms independently selected from N, O and S; and which aromatic rings, if there are 2 or more, can be fused or linked by one or more covalent bond, and which aromatic rings are optionally substituted by 1, 2 or 3 substituents independently selected from F, Cl, CN, OH, C₁₋₆ alkyl, C₁₋₆ alkylthio, per-fluoro-C₁₋₆ alkyl, perfluoro-C₁₋₆ alkylthio, perfluoro-C₁₋₆ alkoxy, C₁₋₆ alkoxy, SO₂R⁴, NR⁵R⁶, NHSO₂R⁷, SO₂NR⁸R⁹, CONR¹⁰R¹¹ and NHCOR¹²;
R⁴, R⁵, R⁶, R⁷, R⁸, R⁹, R¹⁰, R¹¹ and R¹² are each independently H or C₁₋₆ alkyl (optionally substituted by 1 to 3 fluorine atoms) and the pharmaceutically acceptable salts, solvates (including hydrates), and prodrugs thereof.

Preferably R¹ is a phenyl group, optionally substituted by one or two substituents independently selected from F, Cl, C₁₋₄ alkyl, C₁₋₄ alkylthio and C₁₋₄ alkoxy, or a tetrahydropyranyl group. More preferably R¹ is a phenyl group (optionally substituted by F, Cl, methoxy or ethoxy) or a tetrahydropyranyl group. Yet more preferably R¹ is 4-chlorophenyl, 4-fluorophenyl, phenyl, 3-chlorophenyl, 2-ethoxyphenyl, 2-methoxyphenyl, 3-methoxyphenyl, 3-ethoxyphenyl, 4-methoxyphenyl or 4-ethoxyphenyl. Yet more preferably R¹ is 4-chlorophenyl or 4-fluorophenyl. Most preferably R¹ is 4-fluorophenyl.

In an alternative embodiment R¹ is selected from the values associated with the Examples below.

Preferably X represents a direct link.

Preferably Z is

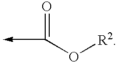

More preferably Z is CO₂H.

Preferably Ar is a biphenyl, pyridinylphenyl, or naphthyl group, optionally substituted by 1, 2 or 3 substituents independently selected from F, Cl, CN, OH, C₁₋₆ alkyl, C₁₋₆ alkylthio, per-fluoro-C₁₋₆ alkyl, perfluoro-C₁₋₆ alkylthio, perfluoro-C₁₋₆ alkoxy, C₁₋₆ alkoxy, SO₂R⁴, NR⁵R⁶, NHSO₂R⁷, SO₂NR⁸R⁹, CONR¹⁰R¹¹ andNHCOR¹². More preferably Ar is a biphenyl, pyridinylphenyl, or naphthyl group, optionally substituted by 1, 2 or 3 substituents independently selected from F, Cl, CN, C₁₋₆ alkyl and C₁₋₆ alkoxy. Yet more preferably Ar is a biphenyl, pyridinylphenyl, or naphthyl group, substituted by F, Cl, CN, methoxy or ethoxy.

Further more preferably Ar is selected from

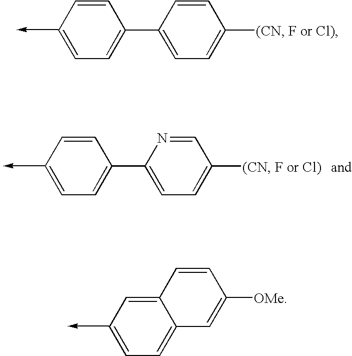

Further yet more preferably Ar is selected from

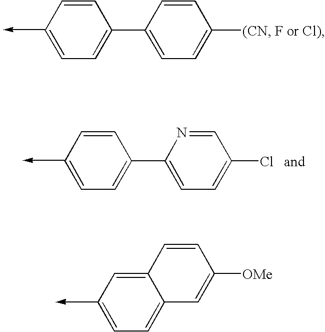

Further yet more preferably Ar is selected from

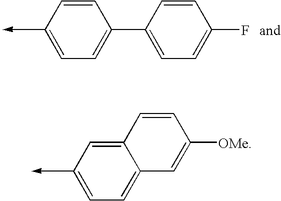

Most preferably Ar is represented by

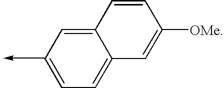

A more preferred group of compounds, salts, solvates and prodrugs are the compounds of the Examples below (especially Examples 2, 5, 6, 10, 14 and 16; more especially Examples 2 and 14); and their salts, solvates, hydrates, and prodrugs.

Pharmaceutically acceptable derivatives of the compounds of formula (I) according to the invention include salts, solvates, complexes, polymorphs, prodrugs, stereoisomers, geometric isomers, tautomeric forms, and isotopic variations of compounds of formula (I). Preferably, pharmaceutically acceptable derivatives of compounds of formula (I) comprise salts, solvates, esters and amides of the compounds of formula (I). More preferably, pharmaceutically acceptable derivatives of compounds of formula (I) are salts, solvates and prodrugs. More preferably, pharmaceutically acceptable derivatives of compounds of formula (I) are salts and solvates.

The pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemi-salts of acids and bases may also be formed, for example, hemi-sulphate and hemicalcium salts.

For a review on suitable salts, see “Handbook of Pharmaceutical Salts: Properties, Selection, and Use” by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The routes below, including those mentioned in the Examples and Preparations, illustrate methods of synthesising the compounds of formula (I). The skilled person will appreciate that the compounds of the invention, and intermediates thereof, could be made by methods other than those specifically described herein, for example by adaptation of the described methods or by methods known in the art. Examples of suitable guides to synthesis, functional group interconversions, use of protecting groups, etc., are: “Comprehensive Organic Transformations” by R C Larock, VCH Publishers Inc. (1989); Advanced Organic Chemistry” by J. March, Wiley lnterscience (1985); “Designing Organic Synthesis” by S Warren, Wiley lnterscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and P G M Wuts, John Wiley and Sons, Inc. (1999); and “Protecting Groups” by P J, Kocienski, Georg Thieme Verlag (1994); and any updated versions of said standard works.

In the general synthetic methods below, unless otherwise specified, the substituents R¹, X, Z and Ar are as defined with reference to the compounds of formula (I) above.

The routes below illustrate methods of synthesising the compounds of formula (I). The skilled person will appreciate that other methods may be equally as viable.

Scheme 1 illustrates the preparation of the compounds of formula (I) via ether formation from intermediates (II) and (III), where LG in (II) is a suitable leaving group. If necessary a suitable base (such as potassium carbonate) and/or additive (such as sodium iodide), and a suitable solvent can be added.

Suitable leaving groups include Cl, Br, I, mesylate, tosylate, etc.

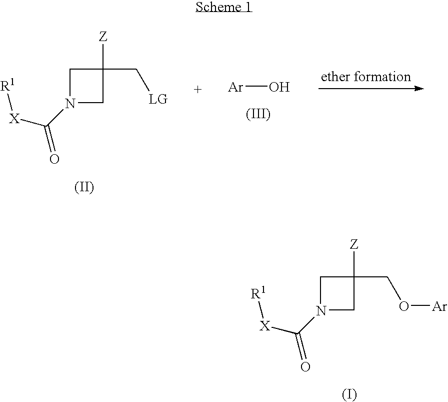

Typical conditions that may be employed involve stirring the azetidine of formula (II) and the hydroxy-aryl compound of formula (III) together with potassium carbonate, cesium carbonate or 1,8-diazobicyclo[5.4.0]undec-7-ene (DBU) in dimethyl sulphoxide (DMSO), dimethylformamide (DMF) or acetonitrile, at a temperature of from 60° C. up to the reflux temperature of the solvent. A suitable alternative is to use an additive (such as sodium iodide or tetrabutyl ammonium iodide) as well as a base. Any suitable high boiling point solvent may be used in place of those mentioned above. At least one equivalent of the intermediate hydroxyaryl compound (III) and at least one equivalent of the base should be used. An excess of one or both may be used if desired. When Z represents C(O)O(C₁₋₆ alkyl) in intermediate (II) and it is desirable for Z to represent CO₂H in the compound of formula (I), the hydrolysis can be done in situ, by adding a suitable base, or water, to the reaction mixture after the ether formation has taken place. Suitable bases for this hydrolysis include lithium hydroxide, or sodium hydroxide.

Scheme 2 illustrates the route used for the preparation of the azetidine intermediates of formula (II) from protected intermediates of formula (IV), wherein PG is a suitable N-protecting group. Any suitable nitrogen protecting group may be used (as described in “Protecting Groups in Organic Synthesis” 3^{rd} edition T. W. Greene and P. G. Wuts, Wiley-Interscience, 1999). Common nitrogen protecting groups (PG) suitable for use include tert-butoxycarbonyl (t-Boc) (which is readily removed by treatment with an acid such as trifluoroacetic acid, or hydrogen chloride in an organic solvent, such as dichloromethane, or 1,4-dioxane), and benzyl (which is readily removed by hydrogenation in the presence of a suitable catalyst, or by treatment with 1-chloroethyl chloroformate).

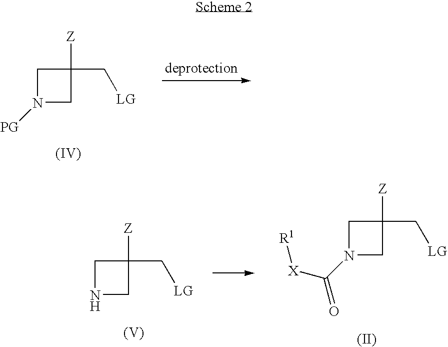

Compounds of formula (IV) can be made by methods well-known to those skilled in the art, for example with reference to literature precedents and/or the Preparations described herein, or by routine adaptation thereof.

The compound of formula (V) can be made by removal of the N-protecting group (PG). For example if PG is a benzyl group, it can be readily removed by hydrogenation in the presence of a suitable catalyst, or by treatment with 1-chloroethyl chloroformate. When X represents a direct link, the C(O)R¹ group may be introduced by acylation of the intermediate compounds of formula (V) using standard acylation chemistry, such as with a suitable (activated) acid (for example an acid chloride R¹COCl or anhydride (R¹CO)₂O) to provide the compounds of formula (II). The acylation is preferably carried out using the acid chloride with a suitable base, such as triethylamine, in a solvent, such as dichloromethane, 1,2 dichloroethane or tetrahydrofuran. Acid chlorides R¹COCl are either commercially available or will be well-known to those skilled in the art with reference to literature precedents.

When X represents —NH—, the C(O)NHR¹ group may be introduced by reaction of the intermediates of formula (V) with a suitable isocyanate R¹NCO to provide the compounds of formula (II). The urea formation is preferably carried out using the isocyanate with a suitable base, such as triethylamine, in a solvent, such as dichloromethane, 1,2 dichloroethane or tetrahydrofuran. Isocyanates R¹NCO are either commercially available or will be well-known to those skilled in the art with reference to literature precedents.

When X represents —O— standard carbamate chemistry may be used to introduce the C(O)OR¹ group. The carbamate formation is preferably carried out using the appropriate chlorocarbonate, R¹O(CO)Cl, and intermediates of formula (V) with a suitable base, such as sodium hydrogen carbonate, in a solvent, such as dichloromethane or 1,2 dichloroethane. Chlorocarbonates R¹O(CO)Cl are either commercially available or will be well-known to those skilled in the art with reference to literature precedents.

The reagents and intermediates of formula (III) are either commercially available or will be well-known to those skilled in the art with reference to literature precedents and/or the Preparations described herein.

Scheme 3 illustrates two alternative routes for the preparation of compounds of formula (I), where the azetidine nitrogen is protected, and C(O)XR¹ is introduced in the final step, or by utilising intermediate alcohol (VI).

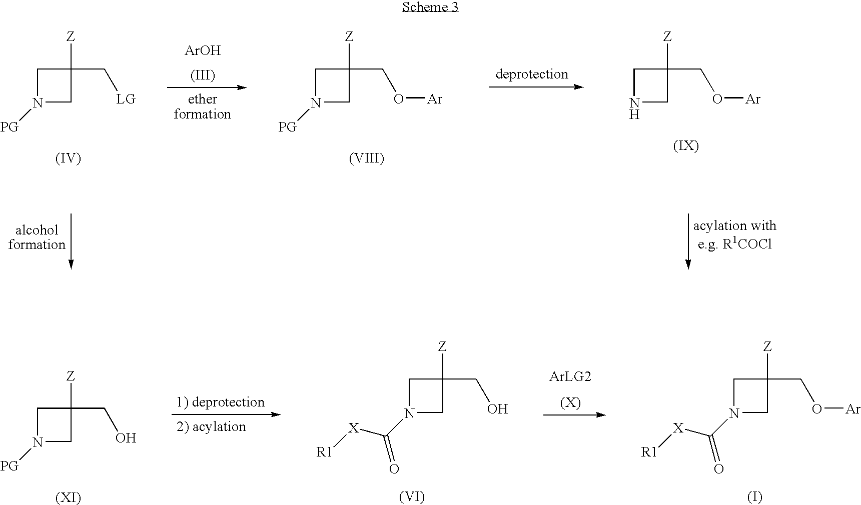

In Scheme 3, the intermediates of formula (IV) and (III) are reacted in an etherification reaction, as previously described in Scheme 1, to provide a protected intermediate (VII), from which the nitrogen protecting group can be removed using standard de-protection strategies, to furnish an intermediate of formula (IX). Any suitable nitrogen protecting group may be used (“Protecting Groups in Organic Synthesis” 3^{rd} edition T. W. Greene and P. G. Wuts, Wiley-Interscience, 1999). Common nitrogen protecting groups (PG) suitable for use include tert-butoxycarbonyl (t-Boc) (readily removed by treatment with an acid, such as trifluoroacetic acid, or hydrogen chloride, in an organic solvent, such as dichloromethane, or 1,4-dioxane), and benzyl (readily removed by hydrogenation in the presence of a suitable catalyst, or by treatment with 1-chloroethyl chloroformate). The C(O)XR¹ group may be introduced by acylation of the deprotected intermediate (IX), as per Scheme 2. This can be done, preferably, via an acid chloride with a suitable base, such as triethylamine, in a solvent, such as dichloromethane, 1,1 dichloroethane, or tetrahydrofuran.

Alternatively, compounds of formula (I) may be prepared from an alcohol of formula (VI), where the Ar group may be introduced by displacement of a suitable leaving group, for example, from an aromatic precursor of formula (X) where LG2 is a suitable leaving group. Suitable leaving groups include F, Cl, Br and I. The displacement reaction involves stirring the alcohol (VI) and a suitable base, preferably sodium hydride, in a suitable solvent, preferably dimethyl sulphoxide, then adding the intermediate (X) and stirring at room temperature. The intermediates of formula (X) are either commercially available or will be well-known to those skilled in the art with reference to literature precedents.

Intermediates of formula (VIl) can be made from intermediates of formula (V) described in Scheme 2, where the azetidine can be protected with a suitable nitrogen protecting group (PG) as mentioned above. The preferred protecting groups are t-Boc or benzyl.

Compounds of formula (VI) can be prepared from intermediates of formula (XI), in a similar manner to the preparation described for intermediates of formula (II), where deprotection followed by acylation provides the product.

Scheme 4 illustrates a route for preparation of the alcohol intermediate (XI) from the azetidine intermediate of formula (IV), via acetate (XII).

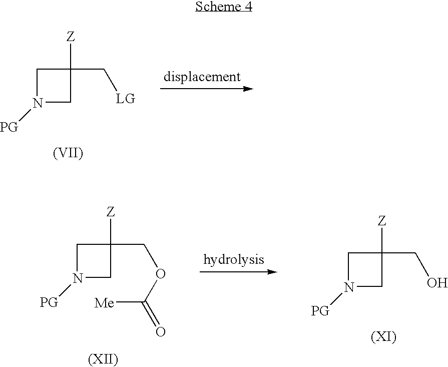

Intermediates of formula (VIl) can be converted into acetates of formula (XII) by stirring compounds of formula (VII) with a suitable metal acetate in order to displace the leaving group (LG). The preferred method is to use cesium acetate, with sodium iodide as an additive, in dimethyl sulphoxide, with heating. Intermediates (XII) can be converted into alcohols (XI) by hydrolysis of the acetates, using a suitable base in a polar organic solvent, preferably potassium carbonate in ethanol.

Alternatively, compounds of formula (I), having particular Ar groups, may be converted into other compounds of formula (I). For example:
i) Compounds of formula (Ia), where Ar contains a suitable leaving group LG3, such as bromo or chloro, can be converted into compounds of formula (Ib), as shown in Scheme 5, e.g. by Suzuki coupling with an appropriate “Ar2-boronic acid” under standard Suzuki coupling conditions.

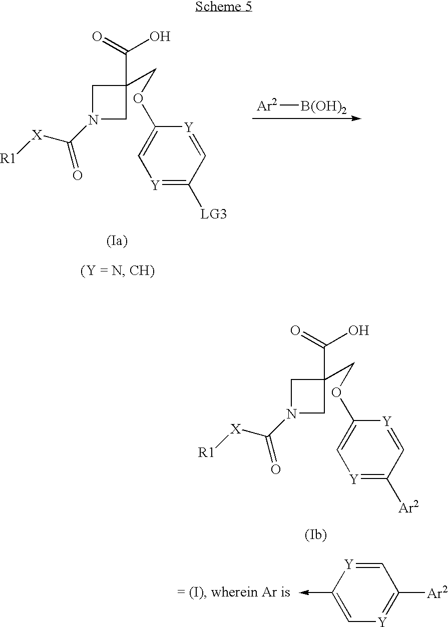

ii) Certain compounds of formula (I) can be converted into certain other compounds of formula (I) by functional group transformations, for example by transformation of the “Z” moiety (Scheme 6) For example, compounds of formula (I), where Z is CO₂H can be converted into acyl sulphonamides (where Z is CONHSO₂R³), via amides (XIII). The acid is suitably activated, then ammonia is added in order to provide an amide (XIII). Activation with ethyl chloroformate in a suitable solvent, such as dichloromethane, is preferred. The amide is then stirred with a suitable base, at a low temperature, then treated with the appropriate sulphonyl compound R³SO₂LG4 (where LG4 is a suitable leaving group such as Cl) to obtain the acyl sulphonamide. Preferred conditions are sodium bis(trimethylsilyl)amide as the base, in tetrahydrofuran as the preferred solvent.

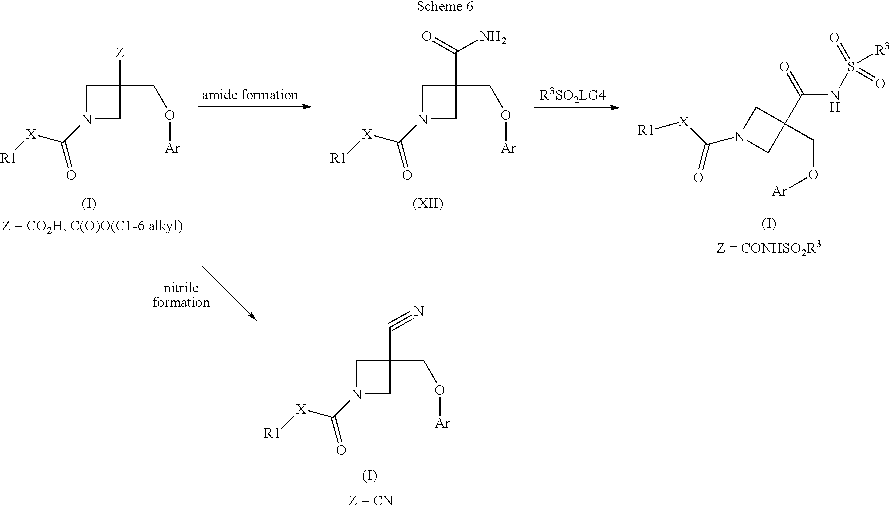

Alternatively compounds of formula (I), where Z is CN, can be prepared from the same acid using a suitable coupling reagent and base with ammonium chloride. Preferred conditions are 1-propyl phosphonic acid cyclic anhydride as the coupling reagent with triethylamine in tetrahydrofuran at reflux.

According to a further embodiment the invention provides novel intermediate compounds of general formula (II), (IV), (V), (VI), (VIII), (IX), (XI), (XII) and (XIII).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. A currently accepted classification system for organic hydrates is one that defines isolated site, channel, or metal-ion coordinated hydrates—see “Polymorphism in Pharmaceutical Solids” by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem. Comm., 17, 1889-1896, by O. Almarsson and M. J. Zaworotko (2004). For a general review of multi-component complexes, see J. Pharm. Sci., 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N—N⁺(CH₃)₃) polar head group. For more information, see Crystals and the Polarizinq Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

As indicated above, so-called ‘prodrugs’ of the compounds of formula (I) are also within the scope of the invention. Thus certain derivatives of compounds of formula (I), which may have little or no pharmacological activity themselves, can be converted into compounds of formula (I) having the desired activity, for example by hydrolytic cleavage, when administered into, or onto, the body. Such derivatives are referred to as ‘prodrugs’. Further information on the use of prodrugs may be found in “Pro-drugs as Novel Delivery Systems”, Vol. 14, ACS Symposium Series (T. Higuchi and W. Stella) and “Bioreversible Carriers in Drug Design”, Pergamon Press, 1987 (Ed. E. B. Roche, American Pharmaceutical Association).

Prodrugs in accordance with the invention can be produced by replacing appropriate functionalities present in the compounds of formula (I) with certain moieties known to those skilled in the art as ‘pro-moieties’ as described, for example, in “Design of Prodrugs” by H. Bundgaard (Elsevier, 1985).

Some examples of prodrugs in accordance with the invention include:
(i) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(ii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Thus within the scope of the invention are envisaged the metabolites of the compounds of formula (I) when formed in vivo.

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism. Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counter ion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

The compounds of the invention may be administered alone or in combination with one or more other compounds of the invention or in combination with one or more other drugs (or as any combination thereof).

The compounds of the present invention may be administered in combination with PDE5 inhibitors. Thus in a further aspect of the invention, there is provided a pharmaceutical product containing an EP2 antagonist and one or more PDEV inhibitors as a combined preparation for simultaneous, separate or sequential use in the treatment of endometriosis.

PDEV inhibitors useful for combining with compounds of the present invention include, but are not limited to:
(i) Preferably 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, e.g., as sold as Viagra®) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methyl piperazine (EP-A-0463756);5-(2-ethoxy-5-morpholinoacetylphenyl)-1-methyl-3-n-propyl-1,6-dihydro-7H-pyrazolo[4,3-d]pyrimidin-7-one (EP-A-0526004);3-ethyl-5-[5-(4-ethylpiperazin-1-ylsulphonyl)-2-n-propoxy phenyl]-2-(pyridin-2-yl)methyl-2,6-dihydro-7H-pyrazolo[4,3-d]pyrimidin-7-one (WO 98/49166);3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo [4,3-d]pyrimidin-7-one(WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (WO99/54333);5-[2-ethoxy-5-(4-ethylpiperazin-1-ylsulphonyl)pyridin-3-yl]-3-ethyl-2-[2-methoxy ethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (WO 01/27113, Example 8);5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d] pyrimidin-7-one(WO 01/27113, Example 15);5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (WO 01/27113, Example 66);5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (WO 01/27112, Example 132); (6R,12aR)-2,3,6,7,12,12a-hexahydro-2-methyl-6-(3,4-methylenedioxyphenyl) pyrazino[2′,1′:6,1]pyrido[3,4-b]indole-1,4-dione (tadalafil, IC-351, Cialis®), i.e. the compound of examples 78 and 95 of published international application WO95/19978, as well as the compound of examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e., the compound of examples 20, 19, 337 and 336 of published international application WO99/24433; the compound of example 11 of published international application WO93/07124 (EISAI); compounds 3 and 14 from Rotella D. P., J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl-2-pyrrolidine propanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide; 4-[(3-chloro-4-methoxybenzyl)amino]-2-[(2S)-2-(hydroxymethyl)pyrrolidin-1 -yl]-N-(pyrimidin-2-ylmethyl)pyrimidine-5-carboxamide (TA-1790); 3-(1-methyl-7-oxo-3-propyl-6,7-dihydro-1H-pyrazolo[4,3-d]pyrimidin-5-yl)-N-[2-(1-methylpyrrolidin-2-yl )ethyl]-4-propoxybenzene sulfonamide (DA 8159) and pharmaceutically acceptable salts thereof.
(ii) 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, mono-sodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furaziocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propyl indole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866.

Preferably the PDEV inhibitor is selected from sildenafil, tadalafil, vardenafil, DA-8159 and 5-[2-ethoxy-5-(4-ethylpiperazin-1-ylsulphonyl)pyridin-3-yl]-3-ethyl-2-[2-methoxy ethyl]-2,6-dihydro-7H-pyrazolo[4,3-d]pyrimidin-7-one. Most preferably the PDE5 inhibitor is sildenafil and pharmaceutically acceptable salts thereof. Sildenafil citrate is a preferred salt.

The compounds of the present invention may be administered in combination with a V1a antagonist. Thus, in a further aspect of the invention, there is provided a pharmaceutical product containing an EP2 receptor antagonist and one or more V1a antagonists as a combined preparation for simultaneous, separate or sequential use in the treatment of endometriosis.

A suitable vasopressin V1a receptor antagonist is, for example, (4-[4-Benzyl-5-(4-methoxy-piperidin-1-ylmethyl)-4H-[1,2,4]triazol-3-yl]-3,4,5,6-tetrahydro-2H-[1,2′]bipyridinyl), which is Example 26 in WO 2004/37809. A further example of a suitable vasopressin V1a receptor antagonist is 8-chloro-5-Methyl-1-(3,4,5,6-tetrahydro-2H-[1,2′]bipyridinyl-4-yl)-5,6-dihydro-4H-2,3,5,10b-tetraazo-benzo[e]azulene, or a pharmaceutically acceptable salt or solvate thereof, which is Example 5 in WO 04/074291.

Further examples of vasopressin V1a receptor antagonists for use with the invention are: SR49049 (Relcovaptan), atosiban (Tractocile®), conivaptan (YM-087), VPA-985, CL-385004, Vasotocin and OPC21268. Additionally, the V1a receptor antagonists described in WO 01/58880 are suitable for use in the invention.

The compounds of the invention may be administered in combination with an agent which lowers estrogen levels, or which antagonises the estrogen receptor. Thus, in a further aspect of the invention, there is provided a pharmaceutical product containing a progesterone receptor antagonist and one or more agents which lower estrogen levels, or antagonise the estrogen receptor, as a combined preparation for simultaneous, separate or sequential use in the treatment of endometriosis.

Agents which lower estrogen levels include gonadotropin releasing hormone (GnRH) agonists, GnRH antagonists and estrogen synthesis inhibitors. Agents which antagonise the estrogen receptor, i.e. estrogen receptor antagonists, include anti-estrogens.

GnRH agonists suitable for the present invention include leuprorelin (Prostap—Wyeth), buserelin (Suprefact—Shire), goserelin (Zoladex—Astra Zeneca), triptorelin (De-capeptyl—Ipsen), nafarelin (Synarel—Searle), deslorelin (Somagard—Shire), and histrelin/supprelin (Ortho Pharmaceutical Corp/Shire).

GnRH antagonists suitable for the present invention include teverelix (also known as antarelix), abarelix (Plenaxis—Praecis Pharmaceuticals Inc.), cetrorelix (Cetrotide—ASTA Medica), and ganirelix (Orgalutran—Organon).

Anti-estrogens suitable for the present invention include tamoxifen, Faslodex (Astra Zeneca), idoxifene (Coombes et al. (1995) Cancer Res. 55, 1070-1074), raloxifene or EM-652 (Labrie, F et al, (2001) J. Steroid Biochem. Mol. Biol., 79, 213).

Estrogen synthesis inhibitors suitable for the present invention include aromatase inhibitors. Examples of aromatase inhibitors include Formestane (4-OH androstenedione), Exemestane, Anastrozole (Arimidex) and Letroxole.

The compounds of the present invention may be administered in combination with an alpha-2-delta ligand. Thus, in a further aspect of the invention, there is provided a pharmaceutical product containing a progesterone receptor antagonist and one ore more alpha-2-delta ligands, as a combined preparation for simultaneous, separate or sequential use in the treatment of endometriosis.

Examples of alpha-2-delta ligands for use in the present invention are those compounds, or pharmaceutically acceptable salts thereof, generally or specifically disclosed in U.S. Pat. No. 4,024,175, particularly gabapentin, EP641330, particularly pregabalin, U.S. Pat. No. 5,563,175, WO-A-97/33858, WO-A-97/33859, WO-A-99/31057, WO-A-99/31074, WO-A-97/29101, WO-A-02/085839, particularly [(1R,5R,6S)-6-(aminomethyl)bicyclo[3.2.0]hept-6-yl]acetic acid, WO-A-99/31075, particularly 3-(1-aminomethyl-cyclohexylmethyl)-4H-[1,2,4]oxadiazol-5-one and C-[1-(1H-tetrazol-5-ylmethyl)-cycloheptyl]-methylamine, WO-A-99/21824, particularly (3S,4S)-(1-aminomethyl-3,4-dimethyl-cyclopentyl)-acetic acid, WO-A-01/90052, WO-A-01/28978, particularly (1α,3α,5α)(3-amino-methyl-bicyclo[3.2.0]hept-3-yl)-acetic acid, EP0641330, WO-A-98/17627, WO-A-00/76958, particularly (3S,5R)-3-aminomethyl-5-methyl-octanoic acid, WO-A-03/082807, particularly (3S,5R)-3-amino-5-methyl-heptanoic acid, (3S,5R)-3-amino-5-methyl-nonanoic acid and (3S,5R)-3-amino-5-methyl-octanoic acid, WO-A-2004/039367, particularly (2S,4S)-4-(3-fluoro-phenoxymethyl)-pyrrolidine-2-carboxylic acid, (2S,4S)-4-(2,3-difluoro-benzyl)-pyrrolidine-2-carboxylic acid, (2S,4S)-4-(3-chlorophenoxy)proline and (2S,4S)-4-(3-fluorobenzyl)proline, EP1178034, EP1201240, WO-A-99/31074, WO-A-03/000642, WO-A-02/22568, WO-A-02/30871, WO-A-02/30881 WO-A-02/100392, WO-A-02/100347, WO-A-02/42414, WO-A-02/32736 and WO-A-02/28881, all of which are incorporated herein by reference.

Preferred alpha-2-delta ligands for use in the combination of the present invention include: gabapentin, pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (1α,3α,5α)(3-amino-methyl-bicyclo[3.2.0]hept-3-yl)-acetic acid, (3S,5R)-3-aminomethyl-5-methyl-octanoic acid, (3S,5R)-3-amino-5-methyl-heptanoic acid, (3S,5R)-3-amino-5-methyl-nonanoic acid, (3S,5R)-3-amino-5-methyl-octanoic acid, (2S,4S)-4-(3-chlorophenoxy)proline and (2S,4S)-4-(3-fluorobenzyl)proline or pharmaceutically acceptable salts thereof.

Further preferred alpha-2-delta ligands for use in the combination of the present invention are (3S,5R)-3-amino-5-methyloctanoic acid, (3S,5R)-3-amino-5-methylnonanoic acid, (3R,4R,5R)-3-amino-4,5-dimethylheptanoic acid and (3R,4R,5R)-3-amino-4,5-dimethyloctanoic acid, and the pharmaceutically acceptable salts thereof.

Particularly preferred alpha-2-delta ligands for use in the combination of the present invention are selected from gabapentin, pregabalin, (3S,5R)-3-amino-5-methyloctanoic acid, (1α,3α,5α)(3-amino-4,5-methyl-bicyclo[3.2.0]hept-3-yl)-acetic acid, (2S,4S)-4-(3-chlorophenoxy)proline and (2S,4S)-4-(3-fluorobenzyl)proline or pharmaceutically acceptable salts thereof.

The compounds of the present invention may be administered in combination with an oxytocin receptor antagonist. Thus, in a further aspect of the invention, there is provided a pharmaceutical product containing a progesterone receptor antagonist and one or more oxytocin antagonists, as a combined preparation for simultaneous, separate or sequential use in the treatment of endometriosis.

Examples of suitable oxytocin receptor antagonists are atosiban (Ferring A B), barusiban (Ferring A B), TT-235 (Northwestern University), and AS-602305 (Serono S A).

The contents of the published patent applications mentioned above, and in particular the general formulae of the therapeutically active compounds of the claims and exemplified compounds therein, are incorporated herein in their entirety by reference thereto.

The compounds of the present invention may also be administered in combination with any one or more of the following:

(i) Aromatase inhibitor;

(ii) Nuclear hormone receptor modulator;

(iii) Angiogenesis inhibitor;

(iv) VEGF inhibitor;

(v) Kinase inhibitor;

(vi) Protein farnesyl transferase inhibitor;

(vii) Prostanoid receptor antagonist;

(viii) Prostaglandin synthetase inhibitor;

(ix) Bioflavanoid;

(x) Alkylating agent;

(xi) Microtobule modulator, e.g. Microtobule stabilizer;

(xii) Topoisomerase I inhibitor;

(xiii) Protease inhibitor;

(xiv) Chemokine receptor antagonist; or

(xv) Neuroendocrine receptor modulators.

Thus, in a further aspect of the invention, there is provided a pharmaceutical product containing a progesterone receptor antagonist and any one or more of the following:

(i) Aromatase inhibitor;

(ii) Nuclear hormone receptor modulator;

(iii) Angiogenesis inhibitor;

(iv) VEGF inhibitor;

(v) Kinase inhibitor;

(vi) Protein farnesyl transferase inhibitor;

(vii) Prostanoid receptor antagonist;

(viii) Prostaglandin synthetase inhibitor;

(ix) Bioflavanoid;

(x) Alkylating agent;

(xi) Microtobule modulator, e.g. Microtobule stabilizer;

(xii) Topoisomerase I inhibitor;

(xiii) Protease inhibitor;

(xiv) Chemokine receptor antagonist; or

(xv) Neuroendocrine receptor modulators,

as a combined preparation for simultaneous, separate or sequential use in the treatment of endometriosis.

Generally, compounds of the invention will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents. Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Consumable oral films are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999). Other means of topical administration include delivery by electroporation, iontophoresis, phonophoresis, sonophoresis and microneedle or needle-free (e.g. Powderject™, Bioject™, etc.) injection. Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin. The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid. Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insulator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μg to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

The compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and a memory aid.

For administration to human patients, the total daily dose of the compounds of the invention is typically in the range <1 mg to 1000 mg depending, of course, on the mode of administration. For example, oral administration may require a total daily dose of from <1 mg to 1000 mg, while an intravenous dose may only require from <1 mg to 500 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As used herein, the terms “treating” and “to treat”, mean to alleviate symptoms, eliminate the causation either on a temporary or permanent basis, or to prevent or slow the appearance of symptoms. The term “treatment” includes alleviation, elimination of causation (either on a temporary or permanent basis) of, or prevention of symptoms and disorders associated with endometriosis and/or uterine leiomyoma. The treatment may be a pre-treatment as well as a treatment at the on-set of symptoms.

The compounds of the present invention should be useful for the treatment of gynaecological symptoms of painful menstruation (dysmenorrhoea), painful intercourse (dyspareunia), painful defaecation (dyzchexia) or micturition (dysuria) provoked by menstruation, chronic pelvic pain (constant or cyclic painful symptoms present for more than six months), excessive menstrual blood loss (menorrhagia), frequent periods (polymenorrhagia) or infrequent or irregular periods (oligoamenorrhoea or amenorrhoea) either occurring in the absence of specific pathology (dysfunctional uterine bleeding and/or primary dysmenorrhoea), or in association with endometriosis, adenomyosis, polycystic ovarian syndrome, or uterine fibroids.

It is intended that the term treatment encompasses not only the management of the pain symptoms associated with the abovementioned conditions, but also modification of the disease progression itself, i.e., a clinically meaningful benefit to the patients is achieved. Modification of disease progression may result in reduction or elimination of pain. More preferably, modification of disease progression may result in reduction or elimination of pain, and prolonged intervals to symptom onset. Even more preferably, modification of disease progression may result in reduction or elimination of pain, prolonged intervals to symptom onset, and reduction in the need of surgery. Most preferably, modification of disease progression may result in reduction or elimination of pain, prolonged intervals to symptom onset, a reduction in the need of surgery, and preserved and/or improved fertility.

The compounds of formula (I) of the present invention have utility as EP2 antagonists in the treatment of various disease states. Preferably said EP2 antagonists exhibit a functional potency at the EP2 receptor expressed as a Ki, lower than about 1000 nM, more preferably lower than 500 nM, yet more preferably lower than about 100 nM and more preferably still lower than about 50 nM wherein said Ki measurement of EP2 functional potency can be carried out using Protocol 1 below.

Using this assay, compounds according to the present invention exhibit a functional potency at the EP2 receptor expressed as a Ki lower than 1000 nM. Preferred compounds herein exhibit functional potency at the EP2 receptor as defined herein before and are selective for EP2 over DP1. Preferably said EP2 antagonists have a selectivity for EP2 over DP1 wherein said EP2 receptor antagonists are at least about 10-times, preferably at least about 20-times, more preferably at least about 30-times, even more preferably at least about 100-times, more preferably still at least about 300-times, even more preferably still at least about 500-times and especially at least about 1000-times more functionally selective for an EP2 receptor as compared with the DP1 receptor wherein said relative selectivity assessments are based on the measurement of DP1 and EP2 functional potencies which can be carried out using the assays described herein. DP1 activity is measured using Protocol 2 below.

Preferably said EP2 antagonists have a selectivity for EP2 over EP4 wherein said EP2 receptor antagonists are at least about 10-times, preferably at least about 30-times, more preferably at least about 100-times, more preferably still at least about 300-times, even more preferably still at least about 500-times and especially at least about 1000-times more functionally selective for an EP2 receptor as compared with the EP4 receptor wherein said relative selectivity assessments are based on the measurement of EP4 and EP2 functional potencies which can be carried out using the assays as described herein. EP4 activity is measured using Protocol 3 below.

Most preferred are EP2 antagonists have a selectivity for EP2 over DP1 and EP4 wherein said EP2 receptors antagonists are at least about 10-times, preferably at least about 30-times, more preferably at least about 100-times, more preferably still at least about 300-times, even more preferably still at least about 1000-times more functionally selective for an EP2 receptor as compared with the DP1 and EP4 receptors.

The compounds of the present invention may be tested in the screens set out below.

1.0 Measurement of in vitro Antagonist Potency (IC₅₀) of Compounds Against Recombinant Human Prostaglandin E2 Receptor in CHO Cells

The prostaglandin E2 (EP-2) receptor is Gs coupled and agonism of the receptor by PGE2 results in activation of intracellular adenylate cyclase enzymes that synthesise the second messenger signalling molecule, adenosine 3′,5′-cyclic monophosphate (cAMP). CHO cells expressing the recombinant human EP-2 receptor are stimulated with PGE2 (5 nM) equivalent to approximately EC50 values to give the maximal cAMP signal. Decreases in cAMP levels following treatment of stimulated recombinant EP-2 cells with potential antagonist compounds were measured and potency (IC₅₀) calculated as follows. A Chinese hamster ovary (CHO) cell line stably transfected with full length cDNA encoding human Prostaglandin E2 was established using standard molecular biology methods. Test compounds were dissolved in dimethyl sulphoxide (DMSO) at 4 mM. 11 point half log unit increment dilution series of test compound were prepared in DMSO then diluted 1 in 40 in a buffer comprised of phosphate buffered saline (PBS) and 0.05% pluronic F-127 surfactant. Freshly cultured cells at 80-90% confluence were harvested and re-suspended in 90% growth media/10% DMSO. The cells were frozen using a planar freezer and stored in frozen aliquots in cryovials in liquid nitrogen until the day of the experiment. A vial of cells was defrosted in a 37° C. water bath for 2 min, then transferred to 10 ml of Dulbecco's Modified Eagle's Medium (DMEM). The cells were then centrifuged for 5 min at 1000 g and the pellet re-suspended at 1,000,000 cells/ml in DMEM. 5,000 cells (5 ul) were added to 5 ul of the compound dilution series in a 384 well assay plate and pre-incubated for 30 min at 37° C. 5 ul of agonist (15 nM PGE2 in PBS to give 5 nM FAC) was added and plates further incubated for 90 min at 37° C. The relative cAMP concentration in each well was then measured using a [symbol] -galactosidase enzyme fragment complementation method purchased in kit form as the Discoverx cAMP II kit from GE Healthcare, UK. The luminescence readings taken from each assay well were converted into percent effect relative to maximum control wells corresponding to a 30 uM of S-5751 (Shionogi, see e.g. U.S. Pat. No. 6,693,203) demonstrated to give a maximal effect. Sigmoidal curves were fitted to plots of log₁₀ inhibitor concentration vs. percent effect. IC₅₀ estimates were determined as the concentration of test compound giving an effect half way between the bottom and top asymptotes of the sigmoidal dose response curve. Each experiment included an IC₅₀ determination for the literature compound as a standard to track assay consistency and allow fair comparison between values obtained in different experiments. The EC50 of PGE2 is used in combination with the ligand concentration in the assay to determine Ki values for antagonist dose responses using the Cheng-Prusoff equation. Consequently an agonist dose response curve is carried out for each experiment using the same incubation as the antagonist plate.

2.0 Measurement of in vitro Antagonist Potency (IC₅₀) of Compounds Against Recombinant Human Prostaglandin D1 Receptor in CHO Cells

The prostaglandin D1 (DP-1) receptor is Gs coupled and agonism of the receptor by PGE2 results in activation of intracellular adenylate cyclase enzymes that synthesise the second messenger signalling molecule, adenosine 3′,5′-cyclic monophosphate (cAMP). CHO cells expressing the recombinant human DP-1 receptor are stimulated with BW245C (10 nM) equivalent to approximately EC70 values to give the maximal cAMP signal. Decreases in cAMP levels following treatment of stimulated recombinant DP-1 cells with potential antagonist compounds were measured and potency (IC₅₀) calculated as follows. A Chinese hamster ovary (CHO) cell line stably transfected with full length cDNA encoding human Prostaglandin D1 was established using standard molecular biology methods. Test compounds were dissolved in dimethyl sulphoxide (DMSO) at 4 mM. 11 point half log unit increment dilution series of test compound were prepared in DMSO then diluted 1 in 40 in a buffer comprised of phosphate buffered saline (PBS) and 0.05% pluronic F-127 surfactant. Freshly cultured cells at 80-90% confluence were harvested and re-suspended in 90% growth media/10% DMSO. The cells were frozen using a planar freezer and stored in frozen aliquots in cryovials in liquid nitrogen until the day of the experiment. A vial of cells was defrosted in a 37° C. water bath for 2 min, then transferred to 10ml of Dulbecco's Modified Eagle's Medium (DMEM). The cells were then centrifuged for 5 min at 1000 g and the pellet re-suspended at 1,000,000 cells/ml in DMEM. 5,000 cells (5 ul) were added to 5 ul of the compound dilution series in a 384 well assay plate and pre-incubated for 30 min at 37° C. 5 ul of agonist (30 nM BW245C in PBS to give 10 nM FAC) was added and plates further incubated for 90 min at 37° C. The relative cAMP concentration in each well was then measured using a [symbol] -galactosidase enzyme fragment complementation method purchased in kit form as the Discoverx cAMP II kit from GE Healthcare, UK. The luminescence readings taken from each assay well were converted into percent effect relative to maximum control wells corresponding to a 30 uM of S-5751, demonstrated to give a maximal effect. Sigmoidal curves were fitted to plots of log₁₀ inhibitor concentration vs. percent effect. IC50 estimates were determined as the concentration of test compound giving an effect half way between the bottom and top asymptotes of the sigmoidal dose response curve. Each experiment included an IC₅₀ determination for the literature compound as a standard to track assay consistency and allow fair comparison between values obtained in different experiments. The EC70 of BW245C is used in combination with the ligand concentration in the assay to determine Ki values for antagonist dose responses using the Cheng-Prusoff equation. Consequently an agonist dose response curve is carried out for each experiment using the same incubation as the antagonist plate.

3.0 Measurement of in vitro Antagonist Potency (IC₅₀) of Compounds Against Recombinant Human Prostaglandin E4 Receptor in CHO Cells

The prostaglandin E4 (EP-4) receptor is Gs coupled and agonism of the receptor by PGE2 results in activation of intracellular adenylate cyclase enzymes that synthesise the second messenger signalling molecule, adenosine 3′,5′-cyclic monophosphate (cAMP). CHO cells expressing the recombinant human EP-4 receptor are stimulated with PGE2 (6nM) equivalent to approximately EC50 values to give the maximal cAMP signal. Decreases in cAMP levels following treatment of stimulated recombinant EP-2 cells with potential antagonist compounds were measured and potency (IC₅₀) calculated as follows.

A Chinese hamster ovary (CHO) cell line stably transfected with full length cDNA encoding human Prostaglandin E4 was established using standard molecular biology methods. Test compounds were dissolved in dimethyl sulphoxide (DMSO) at 4 mM. 11 point half log unit increment dilution series of test compound were prepared in DMSO then diluted 1 in 40 in a buffer comprised of phosphate buffered saline (PBS) and 0.05% pluronic F-127 surfactant. Freshly cultured cells at 80-90% confluence were harvested and re-suspended in 90% growth media/10% DMSO. The cells were frozen using a planar freezer and stored in frozen aliquots in cryovials in liquid nitrogen until the day of the experiment. A vial of cells was defrosted in a 37° C. water bath for 2 min, then transferred to 10 ml of Dulbecco's Modified Eagle's Medium (DMEM). The cells were then centrifuged for 5 min at 1000 g and the pellet re-suspended at 1,000,000 cells/ml in DMEM. 5,000 cells (5 ul) were added to 5 ul of the compound dilution series in a 384 well assay plate and pre-incubated for 30 min at 37° C. 5 ul of agonist (6 nM PGE2 in PBS to give 2 nM FAC) was added and plates further incubated for 90 min at 37° C. The relative cAMP concentration in each well was then measured using a [symbol] -galactosidase enzyme fragment complementation method purchased in kit form as the Discoverx cAMP II kit from GE Healthcare, UK. The luminescence readings taken from each assay well were converted into percent effect relative to maximum control wells corresponding to a 30 uM of 4-{(S)-1-[5-Chloro-2-(4-chloro-benzyloxy)-benzoylamino]-ethyl}-benzoic acid (WO2005105733), demonstrated to give a maximal effect. Sigmoidal curves were fitted to plots of log₁₀ inhibitor concentration vs. percent effect. IC₅₀ estimates were determined as the concentration of test compound giving an effect half way between the bottom and top asymptotes of the sigmoidal dose response curve. Each experiment included an IC₅₀ determination for the literature compound as a standard to track assay consistency and allow fair comparison between values obtained in different experiments. The EC50 of PGE2 is used in combination with the ligand concentration in the assay to determine Ki values for antagonist dose responses using the Cheng-Prusoff equation. Consequently an agonist dose response curve is carried out for each experiment using the same incubation as the antagonist plate.

In-vitro Biological Data

| Example number | EP2 Ki (nM) |
|---|---|
| 1 | 0.9 |
| 2 | 1.1 |
| 3 | 0.7 |
| 4 | 1.6 |
| 5 | 1.9 |
| 6 | 2.3 |
| 7 | 1.4 |
| 8 | 2.3 |
| 9 | 6.6 |
| 10 | 3.1 |
| 11 | 10.5 |
| 12 | 3.3 |
| 13 | 6.0 |
| 14 | 6.5 |
| 15 | 7.0 |
| 16 | 7.5 |
| 17 | 19.5 |
| 18 | 19.7 |
| 19 | 21.2 |
| 20 | 24.4 |
| 21 | 27.7 |
| 22 | 53.9 |
| 23 | 101.0 |
| 24 | 103.0 |
| 25 | 122.0 |
| 26 | 125.0 |
| 27 | 146.0 |
| 28 | 175.0 |
| 29 | 196.0 |
| 30 | 206.0 |
| 31 | 233.0 |
| 32 | 271.0 |
| 33 | 304.0 |
| 34 | 338.0 |
| 35 | 525.0 |
| 36 | 705.0 |
| 37 | 752.0 |
| 38 | 31.4 |
| 39 | 22.5 |
| 40 | 2.0 |
| 41 | 4.9 |
| 42 | 5.0 |
| 43 | 5.2 |
| 44 | 5.6 |
| 45 | 56.7 |
| 46 | 37.2 |
| 47 | 69.3 |
| 48 | 79.7 |
| 49 | 114.0 |
| 50 | 776.0 |
| 51 | 333.0 |
| 52 | 182.0 |
| 53 | 615.0 |
| 54 | 10.9 |
| 55 | 24.7 |
| 56 | 71.3 |
| 57 | 192.0 |
| 58 | 388.0 |
| 59 | 824.0 |
| 60 | 268.0 |
| 61 | 4.6 |
| 62 | 12.4 |
| 63 | 50.6 |
| 64 | 7.5 |
| 65 | 55.6 |
| 66 | 102.0 |
| 67 | 110.0 |
| 68 | 603.0 |
| 69 | 560.0 |
| 70 | 707.0 |
| 71 | 587.0 |
| 72 | 748.0 |

The compounds of the invention may have the advantage that they are more potent, have a longer duration of action, have a broader range of activity, are more stable, have fewer side effects or are more selective, or have other more useful properties than the compounds of the prior art.

Thus the invention provides:
(i) a compound of formula (I) or a pharmaceutically acceptable derivative thereof;
(ii) a process for the preparation of a compound of formula (I) or a pharmaceutically acceptable derivative thereof;
(iii) a pharmaceutical composition including a compound of formula (I) or a pharmaceutically acceptable derivative thereof, together with a pharmaceutically acceptable diluent, carrier or adjuvant;
(iv) a compound of formula (I) or a pharmaceutically acceptable derivative or composition thereof, for use as a medicament;
(v) a compound of formula (I) for use as a medicament for the treatment of a disorder which would benefit from EP2 antagonism.
(vi) the use of a compound of formula (I) or of a pharmaceutically acceptable derivative or composition thereof, for the manufacture of a medicament for the treatment of endometriosis, uterine fibroids, menorrhagia, adenomyosis, primary and/or secondary dysmenorrhoea, or chronic pelvic pain syndrome;
(vii) a compound of formula (I) or of a pharmaceutically acceptable derivative or composition thereof, for use in treating of endometriosis, uterine fibroids, menorrhagia, adenomyosis, primary and/or secondary dysmenorrhoea, or chronic pelvic pain syndrome;
(viii) use as in (vi) where the disease or disorder is endometriosis and/or uterine fibroids; (ix) a compound as in (viii) where the disease or disorder is endometriosis and/or uterine fibroids;
(x) a method of treatment of a mammal to treat endometriosis, uterine fibroids, menorrhagia, adenomyosis, primary and secondary dysmenorrhoea, chronic pelvic pain syndrome including treating said mammal with an effective amount of a compound of formula (I) or with a pharmaceutically acceptable derivative or composition thereof;
(xi) a method as in (x) where the disease or disorder is endometriosis and/or uterine fibroids;
(xii) novel intermediates as described herein;
(xiii) a combination as described herein.
(xiv) a compound, salt, solvate, prodrug, process, method of treatment, combination therapy, intermediate or pharmaceutical composition, substantially as described herein.

The Preparations and Examples that follow illustrate the invention but do not limit the invention in any way. All starting materials are available commercially or described in the literature. All temperatures are in ° C. Flash column chromatography was carried out using Merck silica gel 60 (9385). Thin layer chromatography (TLC) was carried out on Merck silica gel 60 plates (5729). “R_{f}” represents the distance travelled by a compound divided by the distance travelled by the solvent front on a TLC plate. Melting points were determined using a Gallenkamp MPD350 apparatus and are uncorrected. NMR was carried out using a Varian-Unity Inova 400 MHz NMR spectrometer or a Varian Mercury 400 MHz NMR spectrometer. Mass spectroscopy was carried out using a Finnigan Navigator single quadrupole electrospray mass spectrometer or a Finnigan aQa APCI mass spectrometer.

Where it is stated that compounds were prepared in the manner described for an earlier Preparation or Example, the skilled person will appreciate that reaction times, number of equivalents of reagents and reaction temperatures may have been modified for each specific reaction, and that it may nevertheless be necessary, or desirable, to employ different work-up or purification conditions.

The invention is illustrated by the following non-limiting Examples in which the following abbreviations and definitions are used:
APCI atmospheric pressure chemical ionisation mass spectrum
Arbocel filter agent
br broad
Celite® filter agent
δ chemical shift
d doublet
ES electrospray ionisation
HPLC high pressure liquid chromatography
LRMS low resolution mass spectrum
m multiplet
m/z mass spectrum peak
NMR nuclear magnetic resonance
Prep preparation
psi pounds per square inch
q quartet
Rt retention time
s singlet
t triplet
tlc thin layer chromatography
UV ultraviolet

Named compounds herein have been named using ACD Labs Name Software v7.11™.

Where compounds are purified by HPLC, there are three methods used, shown below.

| | Method a | Method b |
|---|---|---|
| Column | Sunfire C18 4.6 × 50 mm id | Xterra 4.6 × 50 |
| | | mm id |
| Temperature | Ambient | Ambient |
| Mobile Phase A | 0.05% formic acid in water | 0.05% ammonia in |
| | | water |
| Mobile Phase B | 0.05% formic acid in acetonitrile | 0.05% ammonia in |
| | | acetonitrile |
| Gradient - Initial | 5% B | 5% B |
| Time 0 mins | 5% B | 5% B |
| Time 3 mins | 98% B | 98% B |
| Time 4 mins | 98% B | 98% B |
| Time 4.1 mins | 5% B | 5% B |
| Time 5 mins | 5% B | 5% B |
| Flow rate | 1.5 ml/min | 1.5 ml/min |
| Injection volume | 5 ul | 5 ul |

| | Method c |
|---|---|
| | Phenomenex |
| | Luna 10 u C18(2) |
| Column | 150 × 21.2 (mm) 10 micron |
| Temperature | Ambient |
| Mobile Phase A | water |
| Mobile Phase B | Acetonitrile |
| Mobile Phase C | 2% formic acid_{(aq)} |
| Gradient - Initial | A = 90% B = 5% C = 5% |
| Time 0.6 mins | A = 90% B = 5% C = 5% |
| Time 8.50 mins | A = 5% B = 90% C = 5% |
| Time 11.50 mins | A = 5% B = 90% C = 5% |
| Time 11.60 mins | A = 90% B = 5% C = 5% |
| Time 14 mins | A = 90% B = 5% C = 5% |
| Flow rate | 25 ml/min |

### EXAMPLE 1

### 1-(4-Chlorobenzoyl)-3-{[(4′-cyanobiphenyl-4-yl)oxy]methyl}azetidine-3-carboxylic acid

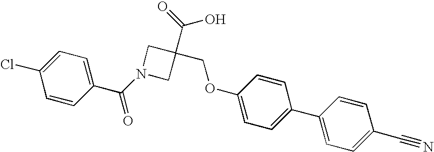

4′-Hydroxy-4-biphenylcarbonitrile (30.9 mg, 0.15 mmol) and potassium carbonate (109 mg, 0.79 mmol) were added to a stirred solution of ethyl 1-(4-chlorobenzoyl)-3-(chloromethyl)azetidine-3-carboxylate (50 mg, 0.15 mmol) (Preparation 8) in dimethyl sulfoxide (3 ml). The resulting mixture was stirred at 130° C. for 0.25 hours. Water (1 mL) was then added and the resulting reaction mixture was heated at 80° C. for 10 mins. it was then allowed to cool before being partitioned between aqueous hydrochloric acid (2M, 5 mL) and dichloromethane (5 mL). The organic layer was dried over sodium sulphate and concentrated under reduced pressure. The residual brown oil was purified by HPLC (method a). LCMS Rt 3.54 mins, ES m/z 446 [M+H]⁺

Examples 2 to 13 were prepared according to the method described above for Example 1, starting from the appropriate compounds of formulae (II) and (III).

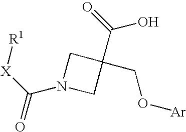

| Ex | R¹ | Ar | Data |
|---|---|---|---|
| 2 | 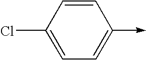 | 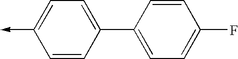 | ¹H NMR (400 MHz, (CD₃)₂CO) δ: 4.22(m, 1 H), 4.38 (m, 1 H), 4.47 (m, 1 H),4.53 (s, 2 H), 4.70 (m, 1 H), 7.05 (d,2 H), 7.42 (d, 2 H), 7.48 (d, 2 H), 7.60 (t,4 H), 7.75 (d, 2 H). APCI MS m/z 440[MH]⁺ |
| 3^{a} | 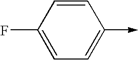 | 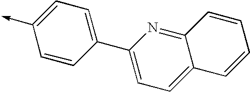 | LCMS Rt 3.06 mins, ES m/z 457[MH]⁺ (Method a) |
| 4 | 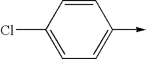 | 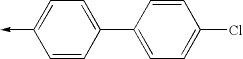 | ¹H NMR (400 MHz, (CD₃)₂CO) δ: 4.23(m, 1 H), 4.39 (m, 1 H), 4.50-4.57 (m,3 H), 4.70 (m, 1 H), 7.03 (d, 2 H), 7.17(t, 2 H), 7.47 (d, 2 H), 7.55 (d, 2 H), 7.60(t, 2 H), 7.75 (d, 2 H). APCI MS m/z456 [MH]⁺ |
| 5^{b} | 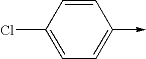 | 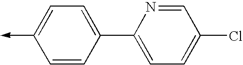 | LCMS Rt 2.39 mins, ES m/z 457[MH]⁺ (Method a) |
| 6^{a} | 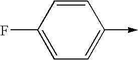 | 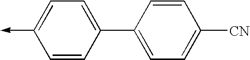 | LCMS Rt 3.55 mins, ES m/z 429 [M −H]⁻ (Method a) |
| 7^{a} | 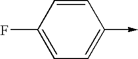 | 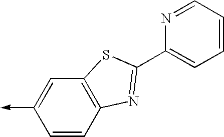 | LCMS Rt 3.38 mins, ES m/z 464[MH]⁺ (Method a) |
| 8^{a} | 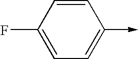 | 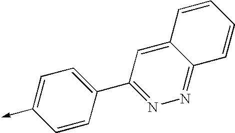 | LCMS Rt 3.13 mins, ES m/z 458[MH]⁺ (Method a) |
| 9^{a} | 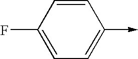 | 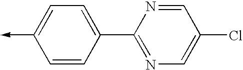 | ¹H NMR (400 MHz, DMSO d6) δ:4.12 (bm, 1 H), 4.27 (bm, 1 H), 4.37(bm, 1 H), 4.47 (s, 2 H), 4.59 (bm, 1 H),7.10 (d, 2 H), 7.29 (m, 2 H), 7.75 (m,2 H), 8.31 (d, 2 H), 8.94 (s, 2 H); LRMSAPCI m/z 442 [MH]⁺ |
| 10^{a} | 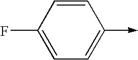 | 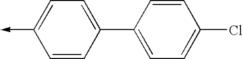 | ¹H NMR (400 MHz, (CD₃)₂CO) δ: 4.22(m, 1 H), 4.40 (m, 1 H), 4.50 (m, 1 H),4.55 (s, 2 H), 4.73 (m, 1 H), 7.10 (d,2 H), 7.22 (t, 2 H), 7.43 (d, 2 H), 7.61 (t,4 H), 7.81 (t, 2 H). ESI MS m/z 438 [M −H]⁻ |
| 11^{a,c} | 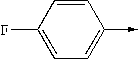 | 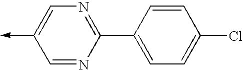 | LCMS Rt 3.58 mins, ES m/z 442[MH]⁺ (Method a) |
| 12^{a,b} | 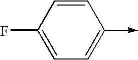 | 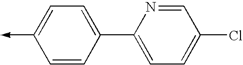 | ¹H NMR (400 MHz, CD₃OD) [symbol] 4.43(d, 1 H), 4.42-4.49 (m, 4 H), 4.66-4.72(d, 1 H), 7.07 (d, 2 H), 7.21 (t, 2 H),7.73-7.86 (m, 4 H), 7.94 (d, 2 H), 8.54(d, 1 H); LRMS ES m/z 441 [MH]⁺ |
| 13 | 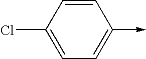 | 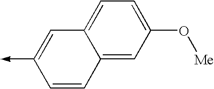 | LCMS Rt 2.76 mins, ES m/z 426[MH]⁺ (Method a) |

^{a}refers to Preparation 5
^{b}refers to Preparation 17
^{c}refers to Preparation 19

### EXAMPLE 14a

### 1-(4-Fluorobenzoyl)-3-{[(6-methoxy-2-naphthyl)oxy]methyl}azetidine-3-carboxylic acid

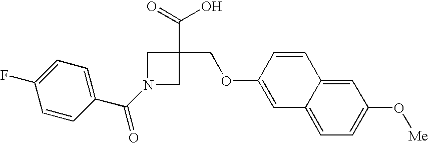

2-Hydroxy-6-methoxynaphthalene (32.3 g, 0.19 mol) and potassium carbonate (51.3 g, 0.37 mol) were added to a stirred solution of ethyl 1-(4-fluorobenzoyl)-3-(chloromethyl)azetidine-3-carboxylate (37.1 g, 0.12 mol) (Preparation 5) in dimethyl sulfoxide (200 mL). The resulting mixture was stirred at 120° C. for 50 minutes. Water (50 mL) was added and the mixture was heated at 80° C. for 1 hour and allowed to cool, before being partitioned between aqueous hydrochloric acid (2M, 1.2 L) and ethyl acetate (1.5 L). The organic layer was dried over sodium sulphate and concentrated under reduced pressure. The residual brown solid was triturated with diethyl ether (1.5 L) and the resulting pink solid was collected by filtration. The solid was purified by column chromatography on silica gel, eluting with 97.5/2.510.25 dichloromethane/methanol/acetic acid, to afford the title compound as a pale pink solid in 77% yield, 39.3 g. ¹H NMR (400 MHz, CD₃OD) δ: 3.84 (s, 3H), 4.27 (d, 1H), 4.42 (m, 2H), 4.44 (s, 2H), 4.67 (d, 1H), 7.07 (m, 2H), 7.20 (m, 4H), 7.63 (m, 2H), 7.72 (m, 2H); ES m/z 410 [M+H]⁺

### EXAMPLE 14b

### 1-(4-Fluorobenzoyl)-3-{[(6-methoxy-2-naphthyl)oxy]methyl}azetidine-3-carboxylic acid

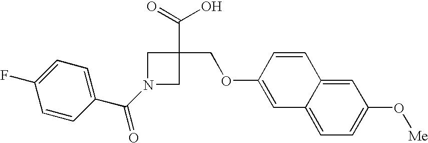

Ethyl 1-(4-fluorobenzoyl)-3-{[(6-methoxy-2-naphthyl)oxy]methyl}azetidine-3-carboxylate (50.0 g, 1114 mmol) (Preparation 33) was suspended in acetonitrile (500 ml) and sodium trimethylsilanolate (14.1 g, 126 mmol) and water (2.05 mL, 114 mmol) were added. The suspension was stirred at ambient temperature for 4 hours. A 10% (v/v) aqueous phosphoric acid solution (100 mL, 171 mmol) was then added and the reaction mixture was allowed to stir for 1 hour at ambient temperature and then a further 2 hours at 0° C. The precipitate was collected, washed twice with water (2×250 mL) and dried under reduced pressure to afford the title compound as a white solid in 85% yield, 39.5 g. ¹H NMR (400 MHz, CD₃OD) δ: 3.84 (s, 3H), 4.27 (d, 1H), 4.42 (m, 2H), 4.44 (s, 2H), 4.67 (d, 1H), 7.07 (m, 2H), 7.20 (m, 4H), 7.63 (m, 2H), 7.72 (m, 2H); ES m/z 410 [M+H]⁺

Examples 15 to 38 were prepared according to the method described above for Example 14, starting from the appropriate compounds of formulae (II) and formula (III).

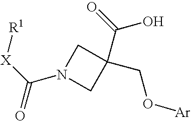

| Ex | -X-R¹ | A | Data |
|---|---|---|---|
| 15 | 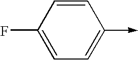 | 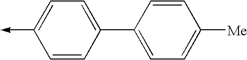 | LCMS Rt 3.76 mins, ES m/z 420[MH]⁺ (Method b) |
| 16 | 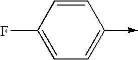 | 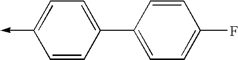 | ¹H NMR (400 MHz, (CD₃)₂CO) δ:4.22 (m, 1 H), 4.40 (m, 1 H), 4.50-4.60(3, 3 H), 4.75 (m, 1 H), 7.10 (d, 2 H),7.17-7.30 (m, 4 H), 7.57-7.66 (m, 4 H),7.82 (t, 2 H). ESI MS m/z 422 [M − H]⁻ |
| 17^{a} | 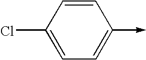 | 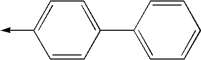 | LCMS Rt 2.54 mins (Method a) |
| 18^{b} | 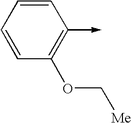 | 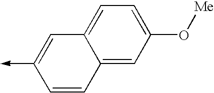 | LCMS Rt 2.30 mins, ES m/z 436[MH]⁺ (Method a) |
| 19 | 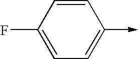 | 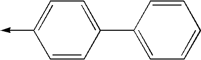 | LCMS Rt 4.32 mins, ES m/z 404[MH]⁺ (Method a) |
| 20^{c} | 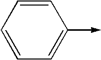 | 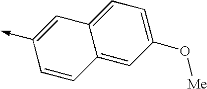 | LCMS Rt 4.32 mins. ES m/z 392[MH]⁺ (Method a) |
| 21^{c} | 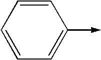 | 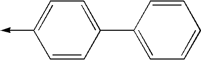 | ¹H NMR (400 MHz, DMSO-d6) δ:4.12 (d, 1 H), 4.28 (d, 1 H), 4.35 (d,1 H), 4.42 (s, 2 H), 4.57 (d, 1 H), 7.04(d, 2 H), 7.31 (t, 1 H), 7.40-7.70 (m,11 H); LRMS APCI m/z 388 [MH]⁺ |
| 22 | 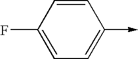 | 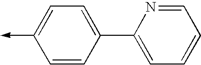 | LCMS Rt 2.52 mins, ES m/z 407[MH]⁺ (Method a) |
| 23^{a} | 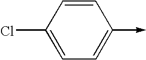 | 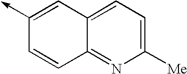 | LCMS Rt 2.47 mins, ES m/z 411[MH]⁺ (Method a) |
| 24 | 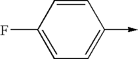 | 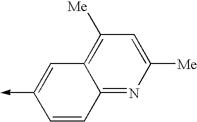 | LCMS Rt 2.14 mins, ES m/z 409[MH]⁺ (Method a) |
| 25 | 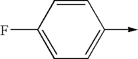 | 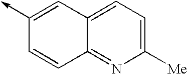 | LCMS Rt 2.09 mins, ES m/z 395[MH]⁺ (Method a) |
| 26^{d} | 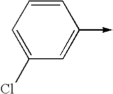 | 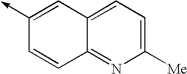 | LCMS Rt 2.17 mins, ES m/z 411[MH]⁺ (Method a) |
| 27 | 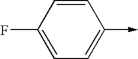 | 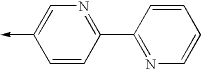 | LCMS Rt 2.44 mins, ES m/z 408[MH]⁺ (Method a) |
| 28^{c} | 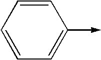 | 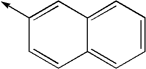 | ¹H NMR (400 MHz, CD₃OD) δ: 4.30(d, 1 H), 4.41 (d, 1 H), 4.46 (d, 1 H),4.46 (s, 2 H), 4.65 (d, 1 H), 7.10 (dd,1 H), 7.25-7.76 (m, 11 H);LRMS APCI m/z 362 [MH]⁺ |
| 29 | 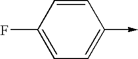 | 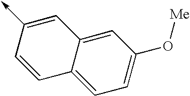 | LCMS Rt 2.99 mins, ES m/z 410[MH]⁺ (Method a) |
| 30 | 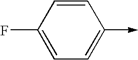 | 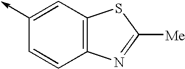 | LCMS Rt 2.99 mins, ES m/z 401[MH]⁺ (Method a) |
| 31^{e} | 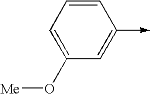 | 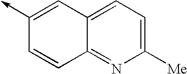 | LCMS Rt 2.09 mins, ES m/z 407[MH]⁺ (Method a) |
| 32^{c} | 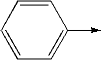 | 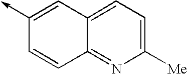 | ¹H NMR (400 MHz, DMSO-d6) δ:2.61 (s, 3 H), 4.14 (bm, 1 H), 4.29 (bm,1 H), 4.36 (bm, 1 H), 4.48 (s, 2 H), 4.58(bm, 1 H), 7.29-7.39 (m, 3 H), 7.44-7.56 (m, 3 H), 7.68 (d, 2 H), 7.81 (d,1 H), 8.12 (d, 1 H); LRMS APCI m/z377 [MH]⁺ |
| 33 | 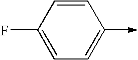 | 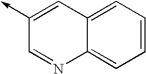 | LCMS Rt 2.92 mins, ES m/z 381[MH]⁺ (Method b) |
| 34^{f} | 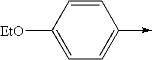 | 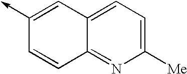 | ¹H NMR (400 MHz, DMSO-d6) δ:1.33 (t, 3 H), 2.61 (s, 3 H), 4.08 (q,2 H), 4.12 (bm, 1 H), 4.27 (bm, 1 H),4.39 (bm, 1 H), 4.49 (s, 2 H), 4.60 (bm,1 H), 6.97 (d, 2 H), 7.29-7.40 (m, 3 H),7.64 (d, 2 H), 7.81 (d, 1 H), 8.13 (d,1 H); LRMS APCI m/z 421 [MH]⁺ |
| 35^{a} | 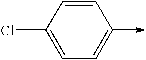 | 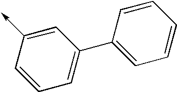 | LCMS Rt 2.44 mins, ES m/z 422[MH]⁺ (Method a) |
| 36^{c} | 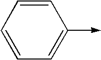 | 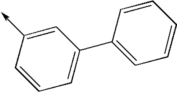 | LCMS Rt 2.37 mins, ES m/z 388[MH]⁺ (Method a) |
| 37 | 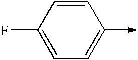 | 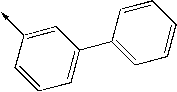 | LCMS Rt 2.38 mins, ES m/z 406[MH]⁺ (Method a) |
| 38^{a} | 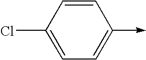 | 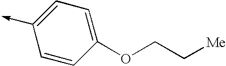 | ¹H NMR (400 MHz, DMSO-D6) [symbol] :0.96 (t, 3 H), 1.68 (m, 2 H), 3.82 (t,2 H), 4.06 (d, 1 H), 4.41 (d, 1 H), 4.50(m, 3 H), 4.55 (d, 1 H), 6.83 (s, 4 H),7.51 (d, 2 H), 7.66 (d, 2 H), 13.5 (br s,1 H); LRMS APCI m/z 404 [MH]⁺ |

^{a}refers to Preparation 8
^{b}refers to Preparation 23
^{c}refers to Preparation 6
^{d}refers to Preparation 22
^{e}refers to Preparation 21
^{f}refers to Preparation 7

### EXAMPLE 39

### 3-{[(2′-Chlorobiphenyl-4-yl)oxy]methyl}-1-(4-fluorobenzoyl)azetidine-3-carboxylic acid

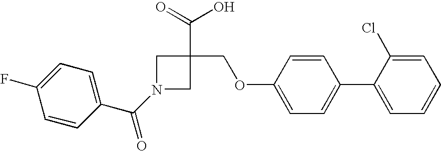

2-Chlorophenylboronic acid (23 mg, 0.147 mmol), cesium carbonate (50 mg, 0.147 mmol) and then tetrakis(triphenylphosphine)palladium(0) (8.5 mg, 0.007 mmol) were added to a stirred solution of 3-[(4-bromophenoxy)methyl]-1-(4-fluorobenzoyl)azetidine-3-carboxylic acid (30 mg, 0.07 mmol) (Preparation 9) in 1,4-dioxane and water (1:1, 3 mL). The mixture was heated to 100° C. for 0.5 hours. The reaction mixture was then allowed to cool before being partitioned between diethyl ether (15 mL) and 2M sodium hydroxide (10 mL). The aqueous layer was made acidic with aqueous hydrochloric acid (2N, 15 mL) and extracted with diethyl ether (15 mL). The diethyl ether layer was dried over sodium sulphate and concentrated under reduced pressure. The resulting residue was triturated with diethyl ether/pentane (1:1) to afford the title compound as an off-white solid in 71% yield (22 mg). ¹H NMR (400 MHz, DMSO-d6) δ: 4.12 (d, 1H), 4.28 (d, 1H), 4.37 (d, 1H), 4.43 (s, 2H), 4.60 (d, 1H), (d, 1H), 7.03 (d, 2H), 7.25-7.41 (m, 7H), 7.54 (d, 1H), 7.76 (m, 2H); LRMS APCI m/z 440 [MH]⁺

Examples 40 to 44 were prepared according to the method described above for Example 39, starting from the appropriate aryl halides and the appropriate boronic acids.

| Ex | -X-R¹ | Ar | Data |
|---|---|---|---|
| 40 | 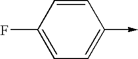 | 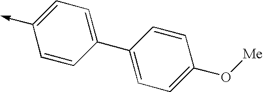 | LCMS Rt 3.48 mins, ES m/z 434[MH]⁺ (Method a) |
| 41 | 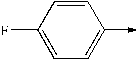 | 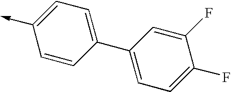 | LCMS Rt 3.69 mins, ES m/z 441[MH]⁺ (Method a) |
| 42^{a} | 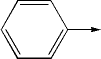 | 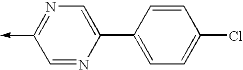 | ¹HNMR (400 MHz, CDCl₃). [symbol] : 4.37(m, 2 H), 4.58 (d, 1 H), 4.79 (m, 3 H),7.40-7.54 (m, 5 H), 7.67 (d, 2 H), 7.82(d, 2 H), 8.30 (s, 1 H), 8.46 (s, 1 H);LRMS APCI m/z 424 [MH]⁺ |
| 43 | 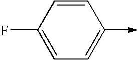 | 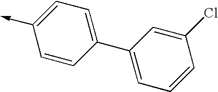 | LCMS Rt 3.81 mins, ES m/z 440[MH]⁺ (Method a) |
| 44 | 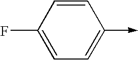 | 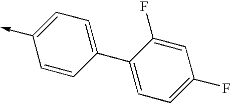 | LCMS Rt 3.67 mins, ES m/z 442[MH]⁺ (Method a) |

^{a}refers to Preparation 10

### EXAMPLE 45

### 1-Benzoyl-3-{[(5-phenylpyrazin-2-yl)oxy]methyl}azetidine-3-carboxylic acid

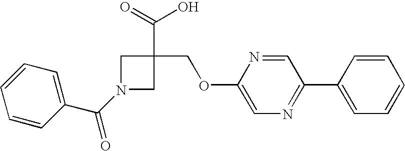

Sodium hydride (11 mg, 0.28 mmol) was added to anhydrous dimethyl sulphoxide (1 mL) at room temperature and stirred, under a nitrogen atmosphere, for 30 minutes. A solution of 1-benzoyl-3-(hydroxymethyl)azetidine-3-carboxylic acid (30 mg, 0.13 mmol) (Preparation 15) in dimethyl sulphoxide (1 mL) was added drop-wise and the mixture was stirred, at room temperature, for 15 minutes. 2-Chloro-5-phenylpyrazine (27 mg, 0.14 mmol) was added and the mixture was stirred, at room temperature, for 4 hours. The mixture was diluted with water (15 mL) and washed with diethyl ether (2×15 mL). The aqueous layer was made acidic with aqueous hydrochloric acid (2N, 2 mL) and extracted with diethyl ether (3×15 mL). The combined ether layers were dried over sodium sulphate and concentrated under reduced pressure. The resulting residue was purified by chromatography on silica gel eluting with ethyl acetate:methanol:acetic acid (95:5:1) to give the title compound, after being crystallised from hot diethyl ether, as a white solid in 60% yield (30 mg). ¹H NMR (400 MHz, DMSO-D6) [symbol] 4.16 (d, 1H), 4.27 (d, 1H), 4.40 (d, 1H), 4.51(d, 1H), 4.74 (s, 2H), 7.4-7.56 (m, 6H), 7.68 (d, 2H), 8.01 (d, 2H), 8.38 (s, 1H), 8.80 (s, 1H), 13.3 (br s, 1H); LRMS APCl m/z 390 [MH]⁺

Examples 46 to 49 were prepared according to the method described above for Example 45, starting from the appropriate alcohols of formula (VI) and the appropriate chlorides.

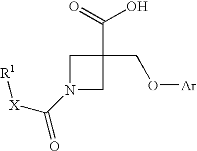

| Ex | -X-R1 | Ar | Data |
|---|---|---|---|
| 46 | 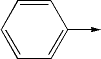 | 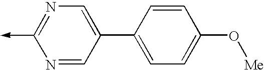 | LCMS Rt 2.69 min, ES m/z 420 [MH]⁺(Method a) |
| 47^{a} | 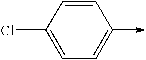 | 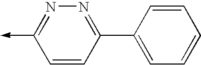 | ¹H NMR (400 MHz, CDCl₃). [symbol] : 4.37(m, 2 H), 4.56 (d, 1 H), 4.75 (d, 1 H),5.01 (s, 2 H), 7.11 (d, 1 H), 7.40 (d,2 H), 7.52 (m, 3 H), 7.62 (d, 2 H), 7.85(d, 1 H), 8.00 (d, 2 H);LRMS APCI m/z 424 [MH]⁺ |
| 48 | 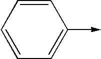 | 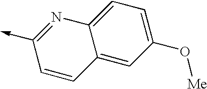 | ¹H NMR (400 MHz, CDCl₃). [symbol] : 3.91(s, 3 H), 4.35 (m, 2 H), 4.51 (d, 1 H),4.75 (d, 1 H), 4.87 (s, 2 H), 6.88 (d,1 H), 7.04 (s, 1 H), 7.25 (d, 1 H), 7.44(m, 4 H), 7.65 (d, 1 H), 7.74 (d, 1 H),7.92 (d, 1 H); LRMS APCI m/z 393[MH]⁺ |
| 49 | 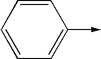 | 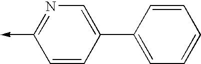 | ¹H NMR (400 MHz, CDCl₃) [symbol] : 4.37(m, 2 H), 4.53 (d, 1 H), 4.73 (d, 1 H),4.80 (s, 2 H), 6.88 (d, 1 H), 7.37-7.57(m, 8 H), 7.68 (d, 2 H), 7.87 (d, 1 H),8.39 (d, 1 H); LRMS APCI m/z 389[MH]⁺ |

^{a}refers to Preparation 16

### EXAMPLE 50

### 1-(4-Fluorobenzoyl)-3-{[(6-methoxy-2-naphthyl)oxy]methyl}-N-(methylsulfonyl)azetidine-3-carboxamide

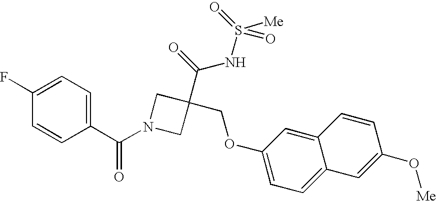

Sodium bis(trimethylsilyl)amide (130 μL of a 1.0M solution in tetrahydrofuran, 0.13 mmol) was added dropwise to a solution of 1-(4-fluorobenzoyl)-3-{[(6-methoxy-2-naphthyl)oxy]methyl}azetidine-3-carboxamide (55 mg, 0.13 mmol) (Preparation 20) in tetrahydrofuran (3 mL) at −60° C. under a nitrogen atmosphere. The resulting mixture was allowed to warm to −40° C. and methane sulfonyl chloride (10.4 μL, 0.13 mmol) was then added. The resulting mixture was allowed to warm to room temperature and quenched with saturated aqueous ammonium chloride solution (1 mL). The mixture was partitioned between diethyl ether (25 mL) and ammonia solution (0.880) (25 mL). The aqueous layer was acidified to pH 1 with 6M HCl and extracted with ethyl acetate (25 mL). The organic extracts were washed with water (20 mL), dried over magnesium sulphate, filtered, and concentrated under reduced pressure. The residue was purified by HPLC (method a). LCMS RT 3.55 min, m/z 485 [M−H]⁻

### EXAMPLE 51

### Ethyl 1-(4-fluorobenzoyl)-3-{[(6-methoxy-2-naphthyl)oxy]methyl}azetidine-3-carboxylate

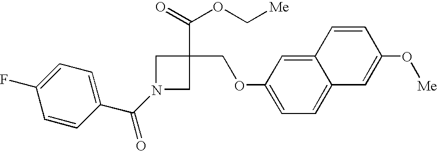

Ethyl chloroformate (38 μL, 0.40 mmol) was added to a stirred suspension of 1-(4-fluorobenzoyl)-3-{[(6-methoxy-2-naphthyl)oxy]methyl}azetidine-3-carboxylic acid (150 mg, 0.36 mmol) (Example 14) in dichloromethane (3 mL). The mixture was stirred for 16 hours. Ammonia solution (0.880) (29 μL, 0.44 mmol) was added and the mixture was stirred for a further 2 hours. It was partitioned with water (20 mL) and extracted with dichloromethane (2×20 mL). The combined organic extracts were dried over sodium sulphate and concentrated under reduced pressure. The residue was purified by chromatography on silica gel, eluting with a gradient of heptane:ethyl acetate (80:20 to 0:100) to afford the title compound as a clear oil in 63% yield (102 mg). ¹H NMR (400 MHz, CDCl₃) δ: 1.27 (t, 3H), 3.91 (s, 3H), 4.28 (q, 2H), 4.44 (m, 6H), 7.12 (m, 6H), 7.64 (m, 2H), 7.72 (m, 2H); LRMS ES m/z 438 [MH]⁺

### EXAMPLE 52

### 1-(4-Fluorobenzoyl)-3-{[(6-methoxy-2-naphthyl)oxy]methyl}azetidine-3-carbonitrile

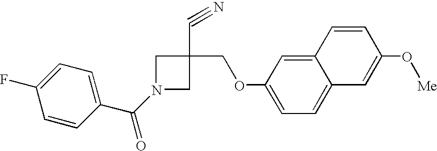

Triethylamine (238 μL, 1.71 mmol), ammonium chloride (39 mg, 0.73 mmol) and 1-propyl phosphonic acid cyclic anhydride (50% by wt. in ethyl acetate, 466 mg, 0.73 mmol) were added to 1-(4-fluorobenzoyl)-3-{[(6-methoxy-2-naphthyl)oxy]methyl}azetidine-3-carboxylic acid (100 mg, 0.24 mmol) (Example 14) in tetrahydrofuran (4 mL). The mixture was heated at reflux for 18 hours. After cooling to room temperature, the mixture was partitioned between ethyl acetate (100 mL) and water (100 mL). The organic extracts were washed with brine (50 mL), dried over magnesium sulphate, filtered, and concentrated under reduced pressure. The residue was triturated with diethyl ether (15 mL). The liquid was decanted and the remaining solid dried under reduced pressure to afford the title compound as an off-white solid in 94% yield (90 mg). LRMS ES 391 [MH]⁺; R_{f} 0.75; DCM/MeOH 95:5.

Example 53 is mentioned below as Preparation 9.

### EXAMPLE 54

### 3-{[(4′-Chlorobiphenyl-4-yl)oxy]methyl}-1-[(4,4-difluorocyclohexyl)carbonyl]azetidine-3-carboxylic acid

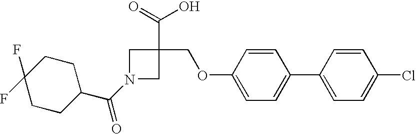

The title compound was prepared according to the method described in Example 1, using ethyl 3-(chloromethyl)-1-[(4,4-difluorocyclohexyl)carbonyl]azetidine-3-carboxylate (22 mg, 0.07 mmol) (Preparation 24) and 4-chloro-4′-hydroxybiphenyl (14 mg, 0.07 mmol) to afford the title compound after HPLC purification. LCMS Rt 2.51 mins, ES m/z 463 [MH]⁺ (Method a)

Examples 55 to 59 were prepared according to the method described above for Example 54, starting from the appropriate compounds of formulae (II) and (III).

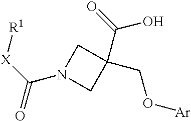

| Ex | -X-R¹ | Ar | Data |
|---|---|---|---|
| 55 | 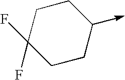 | 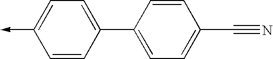 | LCMS Rt 2.44 mins, ES m/z 454[MH]⁺ (Method a) |
| 56 | 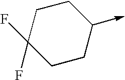 | 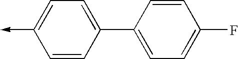 | LCMS Rt 2.46 mins, ES m/z 447[MH]⁺ (Method a) |
| 57 | 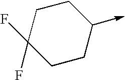 | 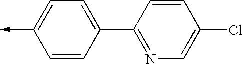 | LCMS Rt 2.61 mins, ES m/z 464[MH]⁺ (Method a) |
| 58^{a} | 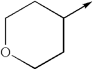 | 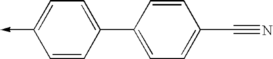 | LCMS Rt 2.22 mins, ES m/z 420[MH]⁺ (Method b) |
| 59^{a} | 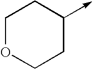 | 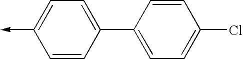 | LCMS Rt 2.44 mins, ES m/z 429[MH]⁺ (Method a) |

^{a}refers to Preparation 25

### EXAMPLE 60

### 1-[(4-Fluorophenoxy)carbonyl]-3-{[(6-methoxy-2-naphthyl)oxy]methyl}azetidine-3-carboxylic acid

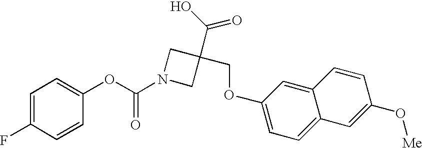

Sodium hydrogen carbonate (43.1 mg, 0.513 mmol) and 4-fluorophenyl chlorocarbonate (19.7 mg, 0.113 mmol) were added to a stirred solution of ethyl 3-{[(6-methoxy-2-naphthyl)oxy]methyl}azetidine-3-carboxylate tosylate (50 mg, 0.10 mmol) (Preparation 28) in dichloromethane (2 mL). The resulting mixture was stirred for 17 hours. It was diluted with dichloromethane (2 mL) and washed with water (2 mL). The layers were separated using a phase separation cartridge and the organic layer was concentrated under reduced pressure to a colourless oil. The oil was dissolved in ethanol (2 mL) and 1 M NaOH (0.10 mmol) was added. The reaction was heated at 70° C. for 1 hour and maintained at room temperature overnight. The solution was acidified with 2M HCl and extracted with ethyl acetate (5 mL), the organic layer was concentrated under reduced pressure and the residue was dissolved in DMSO (1 mL) and purified by HPLC. LCMS Rt 2.97 mins, ES m/z 426 [MH]⁺ (Method a)

Examples 61 and 62 were prepared according to the method described above for Example 60, starting from the appropriate azetidines of formula (IX) and the appropriate chlorocarbonates.

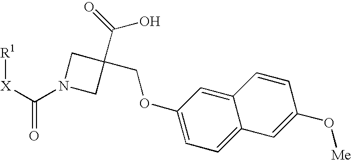

| Ex | -X-R¹ | Data |
|---|---|---|
| 61 | 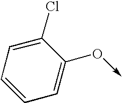 | LCMS Rt 2.39 mins,ES m/z 400 [MH]⁺ (Method a) |
| 62 | 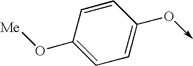 | LCMS Rt 2.29 mins,ES m/z 438 [MH]⁺ (Method a) |

### EXAMPLE 63

### 1-{[(3-Fluorophenyl)amino]carbonyl}-3-{[(6-methoxy-2-naphthyl)oxy]methyl}azetidine-3-carboxylic acid

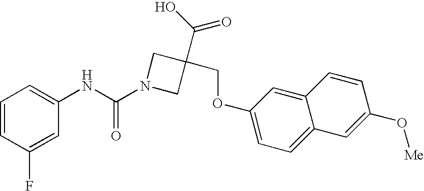

Triethylamine (44.3 μL, 0.318 mmol) and then 1-fluoro-3-isocyanatobenzene (14.1 mg, 0.10 mmol) were added to ethyl 3-{[(6-methoxy-2-naphthyl)oxy]methyl}azetidine-3-carboxylate tosylate (50 mg, 0.10 mmol) (Preparation 28) in dichloromethane (1 mL). The resulting mixture was stirred under nitrogen for 17 hours. The reaction was diluted with dichloromethane (2 mL) and washed with water (2 mL). The layers were separated using a phase separation cartridge and the organic layer was concentrated under reduced pressure. The residue was dissolved in ethanol (2 mL) and 5M NaOH (0.5 mL) was added. The reaction mixture was heated at 70° C. for 1 hour and then stirred at room temperature for 17 hours. The reaction mixture was acidified with 2M HCl and extracted with ethyl acetate (2 mL). The organic layer was concentrated under reduced pressure and the resulting residue was dissolved in DMSO (1 mL) then purified by HPLC. LCMS Rt 2.29 mins, ES m/z 425 [MH]⁺ (Method a)

Examples 64 to 66 were prepared according to the method described above for Example 63, starting from the appropriate azetidines of formula (IX) and the appropriate isocyanates.

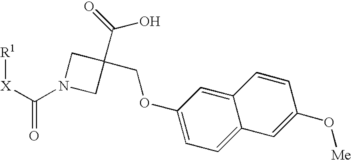

| Ex | -X-R¹ | Data |
|---|---|---|
| 64 | 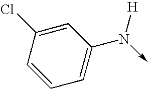 | LCMS Rt 2.36 mins,ES m/z 441 [MH]⁺ (Method a) |
| 65 | 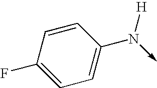 | LCMS Rt 3.37 mins,ES m/z 425 [MH]⁺ (Method b) |
| 66 | 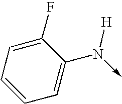 | LCMS Rt 2.24 mins,ES m/z 425 [MH]⁺ (Method a) |

### EXAMPLE 67

### 1-{[(2,3-Dichlorophenyl)amino]carbonyl}-3-[(4-propoxyphenoxy)methyl]azetidine-3-carboxylic acid

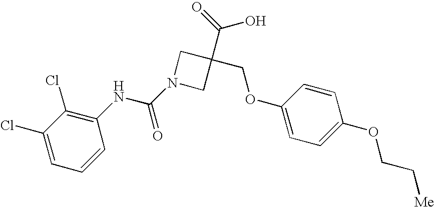

Sodium iodide (33 mg, 0.219 mmol), 4-propoxyphenol (67 mg, 0.438 mmol) and Cs₂CO₃ (360 mg, 1.09 mmol) were added to a stirred solution of ethyl 3-(chloromethyl)-1-{[(2,3-dichlorophenyl)amino]carbonyl}azetidine-3-carboxylate (80 mg, 0.219 mmol) (Preparation 29) in DMF (2 ml). The resulting mixture was stirred at 90° C. for 18 hours. Water (5 ml) and methanol (5 ml) were added and the resulting reaction mixture was stirred at room temperature for 30 minutes. 2M HCl (1.5 ml) was added and the resulting oily suspension was partitioned between water (20 ml) and dichloromethane (20 ml). The dichloromethane layer was dried (Na₂SO₄) and concentrated under reduced pressure to give a dark red oil. The oil was purified by flash chromatography (dichloromethane:methanol:acetic acid 100:0:0 increasing to 90:10:1 as eluant) to give the title compound as a red oil. The oil was purified by HPLC (method c) to give the title compound as an off-white solid in 4% yield (4 mg). ¹H NMR (400 MHz, CD₃OD) δ: 1.03 (t, 3H), 1.72-1.81 (m, 2H), 3.87 (t, 2H), 4.16 (d, 2H), 4.32 (s, 2H), 4.38 (d, 2H), 6.83-6.92 (m, 4H), 7.24-7.34 (m, 2H), 7.65 (m, 1H); LRMS ESCI m/z 453 [M−H]⁻

### EXAMPLE 68

### 3-[(2,3-Dimethylphenoxy)methyl]-1-({[4-(methylthio)phenyl]amino}carbonyl)azetidine-3-carboxylic acid

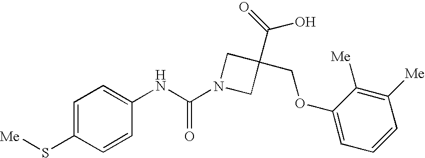

To a solution of ethyl 3-(chloromethyl)azetidine-3-carboxylate hydrochloride (600 μL, 150 μmol of a 0.25M solution in 1,2-dichloroethane) (Preparation 4) was added triethylamine (1300 μL of a 0.25M solution in 1,2-dichloroethane) and 1-isocyanato-4-(methylthio)benzene (650 μL of a 0.25M solution in 1,2-dichloroethane). The vial was capped and vortexed at room temperature for 16 hours. Water (2 mL) was added and the vial was vortexed and centrifuged. The aqueous layer (1.8 mL) was removed and additional saturated aqueous NaHCO₃ (2 mL) was added to the organic layer. This was then vortexed and centrifuged again. The organic layer (1.8 mL) was transferred to a collection vial. 1,2-Dichloroethane (2 mL) was added to the aqueous layer and this was vortexed and centrifuged again. The organic layer (2 mL) was transferred to the collection vial, combining the organic layers. The solvent was removed under reduced pressure. Anhydrous DMF (600 μL) was added to the resulting residue, followed by cesium carbonate (150 mg), sodium iodide solution (600 μL, 150 μmol of a 0.25M solution in anhydrous DMF), and 2,3-dimethylphenol (600 μL, 300 μmol of a 0.5M solution in anhydrous DMF), the vial was capped and shaken at a temperature of 80° C. for 20 hours. The solvent was removed under reduced pressure. Tetrahydrofuran (1000 μL) was added to the vial, followed by methanol (1500 μL) and lithium hydroxide solution (400 μL of a 0.5 M solution in water). The vial was capped and shaken at room temperature for 12 hours. 2M HCl (300 μL) was added and the vial was vortexed. The reaction was concentrated under reduced pressure and the resulting residue dissolved in methanol (800 μL) and water (400 μL) for purification. LRMS ES m/z 401 [MH]⁺

Examples 69 to 72 were prepared according to the method described above for Example 68, starting from the appropriate azetidines of formula (V) and the appropriate isocyanates, followed by the appropriate phenols of formula (III).

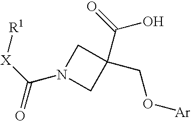

| Ex | -X-R¹ | Ar | Data |
|---|---|---|---|
| 69 | 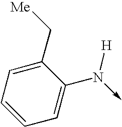 | 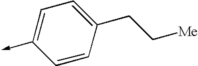 | LRMS ES m/z 397[MH]⁺ |
| 70 | 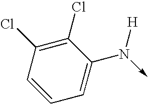 | 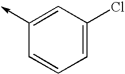 | LRMS ES m/z 430[MH]⁺ |
| 71 | 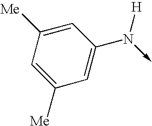 | 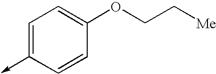 | LRMS ES m/z 413[MH]⁺ |
| 72 | 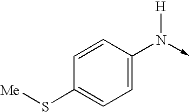 | 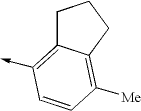 | LRMS ES m/z 427[MH]⁺ |

### Preparation 1: N-Benzyl-3-chloro-2,2-bis(chloromethyl)propanamide

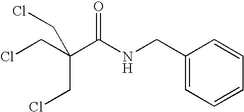

Thionyl chloride (20.5 mL, 283.0 mmol) was added to a solution of 3-chloro-2,2-dichloromethyl propionic acid (48.4 g, 236.0 mmol) in toluene (240 mL). The mixture was stirred at reflux for 17 hours, after which time it was concentrated under reduced pressure and azeotroped with dichloromethane to afford the acid chloride as a cream coloured solid. Triethylamine (49.0 mL, 352.0 mmol) and benzylamine (28.1 mL, 258.0 mmol) were added to a solution of the acid chloride (52.5 g, 234.0 mmol) in toluene (340 mL) at 0° C. The mixture was stirred at room temperature for 3 hours, after which time it was partially concentrated, under reduced pressure, and the solid, which was collected by filtration, was washed with toluene (100 mL), followed by water (500 mL), to afford the title compound as a white solid in 83.1% yield, 57.4 g. ¹H NMR (400 MHz, CDCl₃) δ: 3.92 (s, 6H), 4.52 (d, 2H), 6.20 (s, 1H), 7.33 (m, 5H); LRMS APCI m/z 294 [MH]⁺

### Preparation 2: 1-Benzyl-3,3-bis(chloromethyl)azetidin-2-one

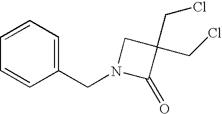

Aqueous sodium hydroxide (10M, 58.5 mL, 585.0 mmol) was added to N-benzyl-3-chloro-2,2-bis(chloromethyl)propanamide (57.4 g, 195.0 mmol) (Preparation 1) and tetrabutylammonium bromide (12.6 g, 39.0 mmol) in dichloromethane (230 mL). The mixture was stirred at room temperature for 2 hours and partitioned between water (500 mL) and dichloromethane (200 mL). The aqueous layer was re-extracted with dichloromethane (50 mL) and the combined organic extracts were washed with water, dried over magnesium sulfate, and concentrated under reduced pressure. The residue was purified by column chromatography on silica gel, eluting with dichloromethane to afford the title compound as a brown oil in 100% yield, 50.5 g. ¹H NMR (400 MHz, CDCl₃) δ: 3.24 (s, 2H), 3.84 (s, 4H), 4.42 (s, 2H), 7.35 (m, 5H); LRMS APCI m/z 258 [MH]⁺

### Preparation 3: Ethyl 1-benzyl-3-(chloromethyl)azetidine-3-carboxylate hydrochloride

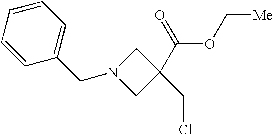

A solution of sodium ethoxide (21% wt. in ethanol, 66.3 mL, 205.0 mmol) in ethanol (70 mL) was added to a solution of 1-benzyl-3,3-bis(chloromethyl)azetidin-2-one (50.4 g, 195.0 mmol) (Preparation 2) in ethanol (210 mL) at 0° C. The mixture was stirred at reflux for 20 hours and then partitioned between water (200 mL) and dichloromethane (300 mL). The aqueous layer was re-extracted with dichloromethane (100 mL) and the combined organic solution was dried over magnesium sulfate and concentrated under reduced pressure to afford an orange oil. A solution of the oil in dichloromethane (100 mL) was treated with a solution of hydrogen chloride in diethyl ether (1M, 250 mL) and the resulting gummy precipitate was triturated with ethyl acetate to afford the title compound as a white solid in 73.2% yield, 43.5 g. ¹H NMR (400 MHz, CD₃OD) δ: 1.32 (t, 3H), 4.11 (s, 2H), 4.31 (m, 4H), 4.47 (m, 4H), 7.51 (m, 5H); LRMS APCI m/z 268 [MH]⁺

### Preparation 4: Ethyl 3-(chloromethyl)azetidine-3-carboxylate hydrochloride

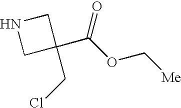

Palladium hydroxide (20% on carbon, 5.7 g) was added to ethyl 1-benzyl-3-(chloromethyl)azetidine-3-carboxylate hydrochloride (57.0 g, 187.4 mmol) (Preparation 3) in ethanol (200 mL) and hydrogenated (30 psi, 60° C.) for 3 hours. The reaction mixture was filtered through Arbocel™ and the filtrate was concentrated under reduced pressure. Trituration of the residue with diethyl ether (100 mL) afforded the title compound as a white solid in 96.0% yield, 38.5 g. ¹H NMR (400 MHz, CDCl₃) δ: 1.30 (t, 3H), 4.05 (d, 2H), 4.19 (s, 2H), 4.25 (d, 2H), 4.30 (q, 2H); LRMS APCI m/z 178 [MH]⁺

### Preparation 5: Ethyl 3-(chloromethyl)-1-(4-fluorobenzoyl)azetidine-3-carboxylate

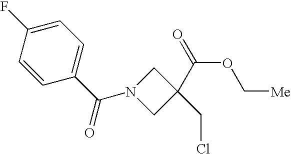

4-Fluorobenzoyl chloride (27.9 g, 176.0 mmol) and triethylamine (53.9 mL, 380.0 mmol) were added to ethyl 3-(chloromethyl)azetidine-3-carboxylate hydrochloride (37.7 g, 176.1 mmol) (Preparation 4) in tetrahydrofuran (300 mL) at 0° C. The reaction mixture was stirred for 2 hours, after which time diethyl ether (200 mL) was added. The mixture was concentrated under reduced pressure to afford the title compound as a red oil in 99.8% yield, 52.7 g. ¹H NMR (400 MHz, CDCl₃) δ: 1.31 (t, 3H), 3.92 (m, 2H), 4.19 (m, 2H), 4.28 (q, 2H), 4.37 (m, 1H), 4.64 (m, 1H), 7.10 (m, 2H), 7.67 (m, 2H)

### Preparation 6: Ethyl 1-benzoyl-3-(chloromethyl)azetidine-3-carboxylate

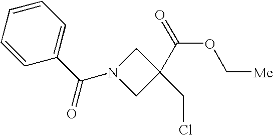

The title compound was prepared according to the method of Preparation 5 using ethyl 3-(chloromethyl)azetidine-3-carboxylate hydrochloride (2.00 g, 7.73 mmol) (Preparation 4) and benzoyl chloride (1.20 g, 8.51 mmol) to afford the title compound as a clear oil in 64% yield, 1.43 g. ¹H NMR (400 MHz, CDCl₃) δ: 1.35 (t, 3H), 3.92 (m, 1H), 4.00 (m, 1H), 4.18 (m, 2H), 4.50 (q, 2H), 4.60 (d, 1H), 4.64 (d, 1H), 7.40-7.54 (m, 3H), 7.65 (d, 2H); LRMS APCI m/z 282 [MH]⁺

### Preparation 7: Ethyl 3-(chloromethyl)-1-(4-ethoxybenzoyl)azetidine-3-carboxylate

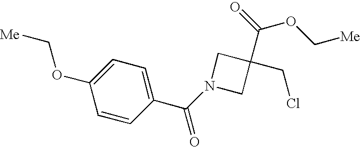

The title compound was prepared according to the method of Preparation 5 using ethyl 3-(chloromethyl)azetidine-3-carboxylate hydrochloride (300 mg, 1.16 mmol) (Preparation 4) and 4-ethoxybenzoyl chloride (214 mg, 1.16 mmol) to afford the title compound as a brown oil in 100% yield, 378 mg. ¹H NMR (400 MHz, CDCl₃) δ: 1.32 (t, 3H), 1.43 (t, 3H), 3.84-4.24 (m, 6H), 4.27 (q, 2H), 4.40 (br s, 1H), 4.64 (br s, 1H), 6.92 (d, 2H), 7.62 (d, 2H); LRMS APCI m/z 326 [MH]⁺

### Preparation 8: Ethyl 1-(4-chlorobenzoyl)-3-(chloromethyl)azetidine-3-carboxylate

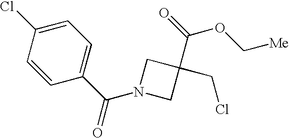

The title compound was prepared according to the method of Preparation 5 using ethyl 3-(chloromethyl)azetidine-3-carboxylate hydrochloride (600 mg, 2.32 mmol) (Preparation 4) and 4-chlorobenzoyl chloride (447 mg, 2.55 mmol) to afford the title compound as a clear oil in 76% yield, 558 mg. ¹H NMR (400 MHz, CDCl₃) δ: 1.36 (t, 3H), 3.85-4.05 (m, 2H), 4.18 (m, 2H), 4.50 (q, 2H), 4.60 (d, 1H), 4.83 (d, 1H), 7.41 (d, 2H), 7.60 (d, 2H); LRMS APCI mlz 316 [MH]⁺

### Preparation 9 (Example 53): 3-[(4-Bromophenoxy)methyl]-1-(4-fluorobenzoyl)azetidine-3-carboxylic acid

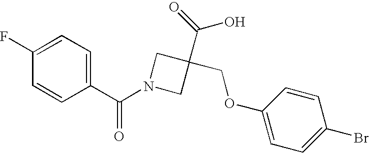

The title compound was prepared according to the method described for Example 1 using ethyl 3-(chloromethyl)-1-(4-fluorobenzoyl)azetidine-3-carboxylate (670 mg, 2.23 mmol) (Preparation 5) and 4-bromophenol (967 mg, 5.59 mmol) to afford the title compound as a pink solid in 55% yield, 503 mg. ¹H NMR (400 MHz, DMSO-d₆) δ: 4.04 (bm, 1H), 4.21 (bm, 1H), 4.25-4.36 (bm, 3H), 4.53 (bm, 1H), 6.88 (d, 2H), 7.24 (bm, 2H), 7.41 (d, 2H), 7.70 (bm, 2H); LRMS APCI m/z 410 [MH]⁺

### Preparation 10: 1-Benzoyl-3-{[(5-chloropyrazin-2-yl)oxy]methyl}azetidine-3-carboxylic acid

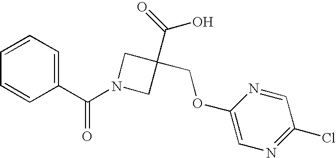

Sodium hydride (95 mg, 2.38 mmol) was added to anhydrous dimethyl sulphoxide (4 mL) at room temperature and stirred, under a nitrogen atmosphere, for 30 minutes. 1-Benzoyl-3-(hydroxymethyl)azetidine-3-carboxylic acid (255 mg, 1.08 mmol) (Preparation 15) in dimethyl sulphoxide (1 mL) was added drop-wise and the resulting mixture was stirred, at room temperature, for 15 minutes. 2,5-Dichloropyrazine (194 mg, 1.3 mmol) was added and the mixture was stirred, at room temperature, for 3 hours. The reaction mixture was diluted with water (15 mL) and washed with diethyl ether (2×15 mL). The aqueous layer was acidified with aqueous hydrochloric acid (2M, 2 mL) and extracted with dichloromethane (2×15 mL). The combined dichloromethane layers were dried over magnesium sulphate and concentrated. The resulting residue was purified by chromatography on silica gel eluting with ethyl acetate:methanol:acetic acid (95:5:1) to give the title compound, as a colourless gum in 80% yield, 305 mg. ¹H NMR (400 MHz, CDCl₃) [symbol] : 4.32 (m, 2H), 4.54 (d, 1H), 4.75 (m, 3H), 7.4-7.56 (m, 3H), 7.65 (d, 2H), 8.04 (s, 1 H), 8.14 (s, 1H); LRMS APCI m/z 348 [MH]⁺

### Preparation 11: Ethyl 3-[(acetyloxy)methyl]-1-benzylazetidine-3-carboxylate

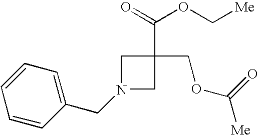

A mixture of acetic acid (163 mL, 2.84 mol) and water (200 mL) was added to a solution of cesium carbonate (307.8 g, 0.945 mol) in water (1 L) under cooling with an ice bath and stirring. The resulting mixture was stirred for 30 minutes at room temperature and concentrated under reduced pressure and azeotroped with 1,4-dioxane (3×300 mL). The residue was dried under high vacuum to give cesium acetate (263.5 g, 1.888 mol) as a white powder. A mixture of this product, ethyl 1-benzyl-3-(chloromethyl) azetidine-3-carboxylate hydrochloride (63.0 g, 0.236 mol) (Preparation 3), and sodium iodide (70.8 g, 0.472 mol) in dimethyl sulphoxide (1 L) was stirred at 95-100° C. for 8 hours. The reaction mixture was cooled to room temperature. Water (1 L) was added, and the product was extracted with hexane/EtOAc mixture (1:1, 5×500 mL). The organic layers were washed with brine, combined, dried with anhydrous sodium sulphate, filtered, and evaporated under reduced pressure. The residue was purified by chromatography on silica gel, eluting with hexane/EtOAc 100:0→50:50 to give the title compound as a light yellow oil in 73% yield, 50.0 g. (R_{f} 0.16; EtOAc/hexane 1:3).

### Preparation 12: Ethyl 1-benzyl-3-(hydroxymethyl)azetidine-3-carboxylate

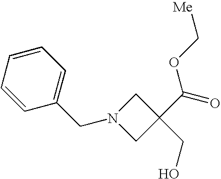

Ethyl 3-[(acetyloxy)methyl]-1-benzylazetidine-3-carboxylate (50.0 g, 172 mmol) (Preparation 11) and potassium carbonate (23.8 g, 172 mmol), in ethanol (1 L), was stirred at room temperature for 6 hours. The reaction mixture was concentrated under reduced pressure and the residue was partitioned between water (800 mL) and chloroform (800 mL). The organic layer was separated, and the aqueous layer was re-extracted with chloroform (2×300 mL). The organic layers were combined, dried with anhydrous sodium sulphate, filtered, and concentrated under reduced pressure. The residue was purified by chromatography on silica gel, eluting with hexane/EtOAc 90:10→50:50 to give the title compound as a light-yellow oil in 50% yield, 21.4 g. (R_{f} 0.1; EtOAc/hexane 1:1).

### Preparation 13: 1-Tert-butyl 3-ethyl 3-(hydroxymethyl)azetidine-1,3-dicarboxylate

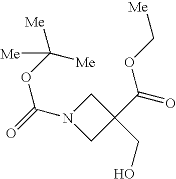

Palladium (5% on carbon, 9.2 g) was added to ethyl 1-benzyl-3-(hydroxymethyl)azetidine-3-carboxylate (21.4 g, 85.8 mmol) (Preparation 12) and di-tert-butyl dicarbonate (19.7 g, 90.0 mmol), in tetrahydrofuran (200 mL). The reaction mixture was hydrogenated (15 psi) for 16 hours. The catalyst was removed by filtration through Celite® and washed with ethanol (5×100 mL). The filtrate was evaporated under reduced pressure. The residue was purified by chromatography on silica gel, eluting with hexane/EtOAc 100:0→75:25 to afford the title compound as a light yellow oil in 67% yield, 15.0 g. (R_{f} 0.33; EtOAc/hexane 1:1); ¹H NMR (400 MHz, DMSO-D6) [symbol] 1.20 (t, 3H), 1.37 (s, 9H), 3.70 (d, 2H), 3.77 (d, 2H), 3.94 (d, 2H), 4.14 (q, 2H), 5.20 (t, 1H).

### Preparation 14: 1-Benzoyl-3-(hydroxymethyl)azetidine-3-carboxylic acid ethyl ester

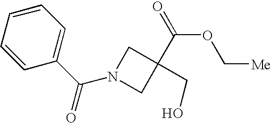

1-Tert-butyl 3-ethyl 3-(hydroxymethyl)azetidine-1,3-dicarboxylate (1.60 g, 6.17 mmol) (Preparation 13) was dissolved in 4M hydrogen chloride in 1,4-dioxane (10 mL) and stirred at room temperature for 18 hours. The reaction mixture was concentrated under reduced pressure, azeotroped with 1,4-dioxane (15 mL) and the resulting residue was dissolved in dichloromethane (15 mL) and triethylamine (1.89 mL, 13.6 mmol) was added. To this mixture was slowly added benzoyl chloride (0.72 mL, 6.17 mmol). The reaction mixture was stirred at room temperature for 2 hours before being concentrated under reduced pressure. The resulting residue was partitioned between diethyl ether (200 mL) and water (100 mL). The organic layer was washed with 1M aqueous hydrochloric acid (50 mL) followed by 2M aqueous sodium hydrogen carbonate (50 mL), dried over magnesium sulphate, filtered and evaporated under reduced pressure to give the title compound as a colourless oil in 86% yield, 1.4 g. LRMS APCI m/z 264 [MH]⁺.

### Preparation 15: 1-Benzoyl-3-(hydroxymethyl)azetidine-3-carboxylic acid

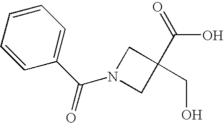

Aqueous sodium hydroxide (2M, 5 mL) was added to 1-benzoyl-3-(hydroxymethyl)azetidine-3-carboxylic acid ethyl ester (1.4 g, 5.3 mmol) (Preparation 14) in methanol (15 mL). The mixture was heated to reflux for 3 hours, cooled and concentrated under reduced pressure. The resulting residue was dissolved in water (10 mL) and washed with diethyl ether (10 mL). The aqueous layer was made acidic with 2M aqueous hydrochloric acid (6 mL) and extracted with ethyl acetate (5×10 mL). The combined ethyl acetate extracts were dried over magnesium sulphate, filtered and concentrated under reduced pressure. The residue was triturated with diethyl ether (5 mL) and filtered to give the title compound as a white solid in 34% yield, 500 mg. ¹H NMR (400 MHz, DMSO-d6) [symbol] 3.35 (s, 1H), 3.72 (s, 2H), 3.98 (d, 1H), 4.11 (d, 1H), 4.18 (d, 1H), 4.40 (d, 1H), 5.20 (br s, 1H), 7.40-7.55 (m, 3H), 7.60 (d, 2H); LRMS APCI m/z 236 [MH]⁺.

### Preparation 16: 1-(4-Chlorobenzoyl)-3-(hydroxymethyl)azetidine-3-carboxylic acid

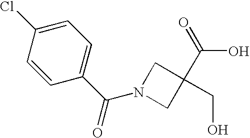

4-Propoxy-phenol (660 mg, 4.35 mmol) was added to ethyl 1-(4-chlorobenzoyl)-3-(chloromethyl)azetidine-3-carboxylate (550 mg, 1.74 mmol) (Preparation 8) and cesium carbonate (2.83 g, 8.70 mmol) in dimethylformamide (5 mL). The resulting mixture was stirred at 80° C. for 6 hours. Water (5 mL) was added and the reaction mixture was heated at 60° C. for 30 minutes and then allowed to cool before being partitioned between water (30 mL) and ethyl acetate (30 mL). The aqueous layer was made acidic with 2M hydrochloric acid and extracted with dichloromethane (2×30 mL). The combined organic extracts were dried over sodium sulphate and concentrated under reduced pressure. The resulting residue was purified by chromatography on silica gel (40 g) eluting with a gradient of dichloromethane:methanol:acetic acid (90:10:1) in dichloro-methane (0% to 100%) to give the title compound as a brown solid in 47% yield, 220 mg. ¹H NMR (400 MHz, DMSO-D6) [symbol] 3.70 (s, 2H), 4.00 (d, 1H), 4.11 (d, 1H), 4.10 (d, 1H), 4.40 (d, 1H), 5.20 (brs, 1H), 7.51(d, 2H), 7.65 (d, 2H), 12.8 (br s, 1H); LRMS APCI m/z 270 [MH]⁺

### Preparation 17: 4-(5-Chloropyridin-2-yl)phenol

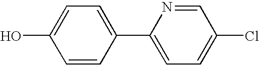

Tetrakis(triphenylphosphine)palladium(0) (4.7 g, 4.05 mmol) was added to a stirred suspension of 2,5-dichloropyridine (12.0 g, 81.1 mmol), 4-hydroxybenzene boronic acid (11.2 g, 81.1 mmol) and potassium carbonate (11.2 g, 81.1 mmol) in dioxane (100 mL) and water (100 mL). The resulting mixture was heated at reflux for 2 hours. The mixture was partitioned between diethyl ether (250 mL) and water (250 mL). The organic phase was washed with brine (150 mL), dried over magnesium sulphate, filtered and concentrated under reduced pressure. The resulting residue was purified by chromatography on silica gel eluting with dichloromethane:methanol (96:4) to afford the title compound as an off-white solid in 81.1% yield, 15.5 g. LRMS ES m/z 204 [M−H]⁻

### Preparation 18: 2-(4-Chlorophenyl)-5-methoxypyrimidine

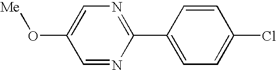

4-Chlorobenzeneboronic acid (7.25 g, 46.35 mmol) was added to a stirred solution of 2-chloro-5-methoxypyrimidine (6.70 g, 46.35 mmol) in 1,4-dioxane (66 mL) and water (33 mL). Potassium carbonate (6.41 g, 46.35 mmol) and tetrakis(triphenylphosphine) palladium(0) (2.68 g, 46.35 mmol) were added and the resulting mixture was stirred at 100° C. for 50 minutes. The mixture was partitioned between diethyl ether (350 mL) and aqueous sodium hydroxide solution (1M, 200 mL). The aqueous layer was re-extracted with diethyl ether (2×100 mL). The combined organic extracts were dried over sodium sulphate, filtered through a plug of silica and concentrated under reduced pressure to afford the title compound, after trituration with diethyl ether (100 mL), as a white solid in 39.6% yield, 4.05 g. LRMS ES m/z 221 [MH]⁺

### Preparation 19: 2-(4-Chlorophenyl)pyrimidin-5-ol

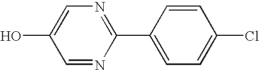

2-(4-Chlorophenyl)-5-methoxypyrimidine (100 mg, 0.453 mmol) (Preparation 18) was dissolved in hydrogen bromide, 30 wt. % in glacial acetic acid (3 mL) and brought to reflux for 3 hours. The mixture was partitioned between diethyl ether (100 mL) and saturated aqueous sodium hydrogen carbonate solution (100 mL). The organic layer was extracted and washed with water (100 mL), dried over magnesium sulphate, filtered and concentrated under reduced pressure. The crude product was triturated with pentane (60 mL) to afford the title compound as an off-white solid in 88.0% yield, 82 mg. ¹H NMR (400 MHz, CDCl₃) δ: 7.42 (d, 2H), 8.29 (d, 2H), 8.45 (s, 2H).

### Preparation 20: 1-(4-Fluorobenzoyl)-3-{[(6-methoxy-2-naphthyl)oxy]methyl}azetidine-3-carboxamide

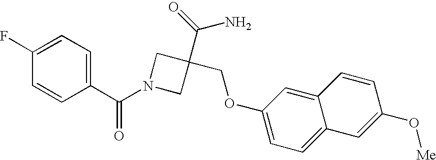

Ethyl chloroformate (38 μL, 0.40 mmol) was added to 1-(4-fluorobenzoyl)-3-{[(6-methoxy-2-naphthyl)oxy]methyl}azetidine-3-carboxylic acid (150 mg, 0.36 mmol) (Example 14) in dichloromethane (3 mL). The mixture was stirred for 16 hours. Ammonia solution (0.880) (29 μL, 0.44 mmol) was added and the mixture was stirred for 2 hours. It was partitioned with water (20 ml) and extracted with dichloromethane (2×20 mL). The combined organic extracts were dried over sodium sulphate and concentrated under reduced pressure. The residue was purified by chromatography on silica gel, eluting with a gradient of heptane:ethyl acetate (80:20 to 0:100) to give a white solid in 42% yield, 63 mg. ¹H NMR (400 MHz, DMSO-d6) δ: 3.81 (s, 3H), 4.13 (d, 1H), 4.20-4.30 (m, 2H), 4.41 (s, 2H), 4.56 (d, 1H), 7.05-7.18 (m, 2H), 7.25-7.37 (m, 5H), 7.60 (s, 1H), 7.64-7.75 (m, 4H); LRMS ES m/z 409 [MH]⁺

### Preparation 21: Ethyl 3-(chloromethyl)-1-(3-methoxybenzoyl)azetidine-3-carboxylate

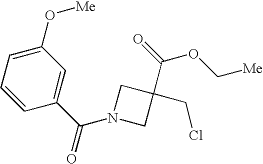

The title compound was prepared according to the method described for Preparation 5 using ethyl 3-(chloromethyl)azetidine-3-carboxylate hydrochloride (Preparation 4) and 3-methoxybenzoyl chloride to afford the title compound. LRMS ES m/z 313 [MH]⁺.

### Preparation 22: Ethyl 1-(3-chlorobenzoyl)-3-(chloromethyl)azetidine-3-carboxylate

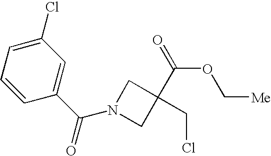

The title compound was prepared according to the method described for Preparation 5 to give the desired compound. LRMS ES m/z 316 [MH]⁺.

### Preparation 23: Ethyl 3-(chloromethyl)-1-(2-ethoxybenzoyl)azetidine-3-carboxylate

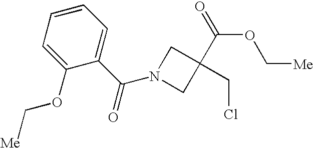

The title compound was prepared according to the method described for Preparation 5 using ethyl 3-(chloromethyl)azetidine-3-carboxylate hydrochloride (Preparation 4) and 3-chlorobenzoyl chloride to afford the title compound. LRMS ES m/z 326 [MH]⁺.

### Preparation 24: Ethyl 3-(chloromethyl)-1-[(4,4-difluorocyclohexyl)carbonyl]azetidine-3-carboxylate

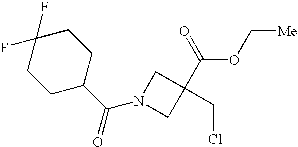

N-[3-(Dimethylamino)propyl]-N′-ethylcarbodiimide hydrochloride (89.5 mg, 0.467 mmol), 4,4-difluorocyclohexanecarboxylic acid (76.7 mg, 0.467 mmol) and triethylamine (195 μL, 1.4 mmol) were added to ethyl 3-(chloromethyl)azetidine-3-carboxylate hydrochloride (100 mg, 0.467 mmol) (Preparation 4) in dichloromethane (5 mL). The reaction was stirred for 1 hour at room temperature and water (5 ml) was added. The resulting mixture was stirred vigorously for 5 minutes. The layers were then separated. The organic layer was washed again with water (5 mL), dried over magnesium sulphate and concentrated under reduced pressure. The resulting gum was purified by passing through an Isolute Flash SCX-2 cartridge, eluting with methanol to give the title compound as a white solid in 59% yield, 89 mg. ¹H NMR (400 MHz, CDCl₃) δ: 1.32 (t, 3H), 1.62-1.95 (m, 6H), 2.13-2.30 (m, 2H), 3.85 (s, 2H), 3.96 (m, 2H), 4.12 (bd, 2H), 4.18 (d, 1H), 4.28 (q, 2H), 4.54 (bd, 1H); LRMS APCl m/z 324/326 [MH]⁺

### Preparation 25: Ethyl 3-(chloromethyl)-1-(tetrahydro-2H-pyran-4-ylcarbonyl)azetidine-3-carboxylate

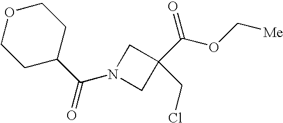

The title compound was prepared according to the method described for Preparation 24 to using ethyl 3-(chloromethyl)azetidine-3-carboxylate hydrochloride (100 mg, 0.47 mmol) (Preparation 4) and tetrahydropyran-4-yl carboxylic acid (50 mg, 0.38 mmol) to afford the title compound as a white solid in 51% yield, 57 mg. ¹H NMR (400 MHz, CDCl₃) δ: 1.27 (t, 3H), 1.55 (bd, 2H), 2.86 (qd, 2H), 2.39 (m, 1H), 3.39 (td, 2H), 3.83 (bd, 1H), 3.88-4.02 (m, 4H), 4.09 (bd, 1H), 4.14 (bd, 1H), 4.26 (q, 2H), 4.52 (bd, 1H); LRMS APCI m/z 290/292 [MH]⁺

### Preparation 26: 1-Tert-Butyl 3-ethyl 3-(chloromethyl)azetidine-1,3-dicarboxylate

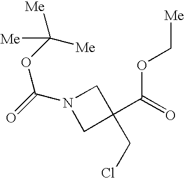

Ethyl 3-(chloromethyl)azetidine-3-carboxylate hydrochloride (4.0 g, 18.68 mmol) (Preparation 4) was suspended in ethyl acetate (60 mL) and triethylamine (5.21 mL, 37.4 mmol) was added, followed by di-tert-butyl dicarbonate (4.49 g, 20.6 mmol). The mixture was stirred under nitrogen for 18 hours. Ethyl acetate (60 ml) was added, and the mixture was washed with water (100 ml), followed by brine (100 ml). The organic phase was separated, dried over magnesium sulphate and concentrated under reduced pressure to give the title compound as a pale yellow oil in 97% yield, 5.01 g. ¹H NMR (400 MHz, CDCl₃) δ: 1.28 (t, 3H), 1.42 (s, 9H), 3.81 (d, 2H), 3.89 (s, 2H), 4.18 (d, 2H), 4.24 (q, 2H); LRMS ES m/z 278 [MH]⁺

### Preparation 27: 1-Tert-Butyl 3-ethyl 3-{[(6-methoxy-2-naphthyl)oxy]methyl}azetidine-1,3-dicarboxylate

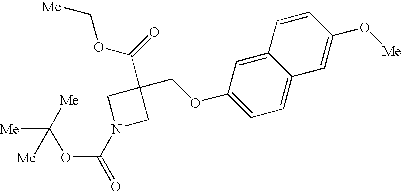

1-Tert-butyl 3-ethyl 3-(chloromethyl)azetidine-1,3-dicarboxylate (200 mg, 0.720 mmol) (Preparation 26) was dissolved in DMSO (5 mL). Potassium carbonate (200 mg, 1.44 mmol), sodium iodide (162 mg, 1.08 mmol) and 6-methoxy-2-naphthol (151 mg, 0.86 mmol) were added and the mixture was heated at 80° C. for 22 hours. The reaction mixture was partitioned between water (10 mL) and ethyl acetate (10 mL). The organic layer was collected and the aqueous layer was further extracted with ethyl acetate (10 mL×2). The combined organic extracts were washed with 1M NaOH (10 mL×2) and water (10 mL×3), dried over sodium sulphate and concentrated under reduced pressure to give the title compound as a dark brown oil in 75% yield, 226 mg. LRMS ES m/z 316 [MH-Boc]⁺

### Preparation 28: Ethyl 3-{[(6-methoxy-2-naphthyl)oxy]methyl}azetidine-3-carboxylate tosylate

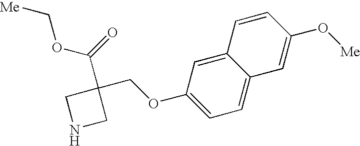

Methanesulphonic acid (308 mg, 3.21 mmol) was added to 1-tert-butyl 3-ethyl 3-{[(6-methoxy-2-naphthyl)oxy]methyl}azetidine-1,3-dicarboxylate (1.11 g, 2.67 mmol) (Preparation 27) in isopropyl acetate (15 mL). The reaction mixture was heated at 40° C. for 18 hours and concentrated under reduced pressure to give the title compound as a red oil in quantitative yield. LRMS ES m/z 316 [MH]⁺

### Preparation 29: Ethyl 3-(chloromethyl)-1-{[(2,3-dichlorophenyl)amino]carbonyl}azetidine-3-carboxylate

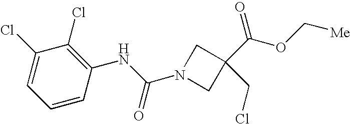

1,2-Dichloro-3-isocyanatobenzene (102 μL, 0.774 mmol), in dichloromethane (2 mL), was added dropwise to ethyl 3-(chloromethyl)azetidine-3-carboxylate hydrochloride (200 mg, 0.774 mmol) (Preparation 4) and triethylamine (226 μL, 1.62 mmol), in dichloromethane (3 mL), at 0° C. The resulting mixture was stirred at room temperature for 18 hours and diluted with dichloromethane (20 mL) and water (20 mL). The slight suspension was filtered and the layers were separated. The aqueous layer was extracted further with dichloromethane (20 mL) and the combined organic extracts were dried over sodium sulphate and concentrated under reduced pressure. The residue was purified by flash chromatography (ethyl acetate:heptane 10:90 increasing to 70:30 as eluant) to give the title compound as a clear oil in 74% yield, 210 mg. ¹H NMR (400 MHz, CDCl₃) δ: 1.34 (t, 3H), 4.00 (s, 2H), 4.05 (d, 2H), 4.31 (q, 2H), 4.41 (d, 2H), 6.70 (bs, 1H), 7.14-7.22 (m, 2H), 8.20 (m, 1H); LRMS ES m/z 365,367 [MH]⁺

### Preparation 30: 1-tert-Butyl 3-ethyl 3-(iodomethyl)azetidine-1,3-dicarboxylate

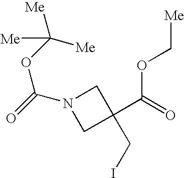

A mixture of 1-tert-butyl 3-ethyl 3-(chloromethyl)azetidine-1,3-dicarboxylate (45.6 g, 164 mmol) (Preparation 26) and sodium iodide (73.8 g, 492 mmol) in acetonitrile (230 mL) was stirred at 80° C. for 18 hours. It was allowed to cool and partitioned between water (180 mL) and ethyl acetate (450 mL). The aqueous layer was separated and the organic layer washed with sodium thiosulfate (23 g, 146 mmol) in water (180 mL), followed by water (180 mL). The organic phase was reduced in volume at atmospheric pressure to afford the title compound as a light-yellow solution in acetonitrile in quantitative yield, 50.4 g in 90 mL acetonitrile. ¹H NMR (400 MHz, CDCl₃) δ: 1.31 (t, 3H), 1.45 (s, 9H), 3.84 (d, 2H), 3.93 (s, 2H), 4.21 (d, 2H), 4.26 (q, 2H).

### Preparation 31: 1-tert-Butyl 3-ethyl 3-{[(6-methoxy-2-naphthyl)oxy]methyl}azetidine-1,3-dicarboxylate

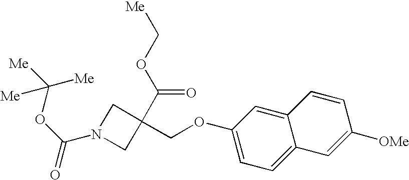

Dimethyl sulfoxide (250 mL), potassium carbonate (37.73 g, 274 mmol) and 6-methoxy-2-naphthol (24.96 g, 143 mmol) were added to a solution of 1-tert-butyl 3-ethyl 3-(iodomethyl)azetidine-1,3-dicarboxylate (50.4 g, 137 mmol) in acetonitrile (90 mL) (Preparation 30). The reaction mixture was then heated at 80° C. for 4 hours, after which time it was allowed to cool before being partitioned between water (500 mL) and ethyl acetate (500 mL). The aqueous layer was separated and the organic layer was washed twice with water (2×500 mL). The acetonitrile was removed at atmospheric pressure and replaced with fresh ethyl acetate (500 mL) to afford the title compound as a solution in ethyl acetate in assumed quantitative yield, 57.0 g. ¹H NMR (400 MHz, CDCl₃) δ: 1.29 (t, 3H), 1.48 (s, 9H), 3.91 (s, 3H), 4.03 (d, 2H), 4.24-4.30 (m, 4H), 4.40 (s, 2H), 7.13 (m, 4H), 7.65 (m, 2H)

### Preparation 32: Ethyl 3-{[(6-methoxy-2-naphthyl)oxy]methyl}azetidine-3-carboxylate tosylate

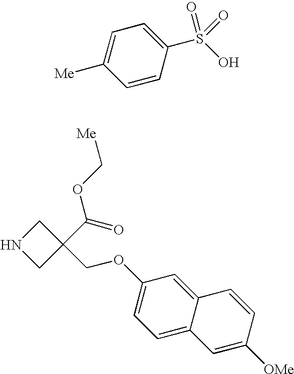

To a solution of 1-tert-butyl 3-ethyl 3-{[(6-methoxy-2-naphthyl)oxy]methyl}azetidine-1,3-dicarboxylate (57.0 g, 137 mmol) in ethyl acetate (500 mL) (Preparation 31) was added p-toluenesulfonic acid monohydrate (32.0 g, 160 mmol). The reaction was stirred at 60° C. for 5.5 hours, after which time it was cooled to 0° C. and granulated for 1 hour. The precipitate was collected and washed twice with ethyl acetate (2×500 mL). It was dried under reduced pressure to afford the title compound as an off-white solid in 73% yield, 49.0 g. ¹H NMR (400 MHz, CDCl₃) δ: 1.22 (t, 3H), 2.28 (s, 3H), 3.84 (s, 3H), 4.12 (d, 2H), 4.22 (q, 2H), 4.32 (d, 2H), 4.46 (s, 2H), 7.11 (d, 2H), 7.14-7.20 (m, 2H), 7.29-7.35 (m, 2H), 7.48 (d, 2H), 7.76 (t, 3H).

### Preparation 33: ethyl 1-(4-fluorobenzoyl)-3-{[(6-methoxy-2-naphthyl)oxy]methyl}azetidine-3-carboxylate

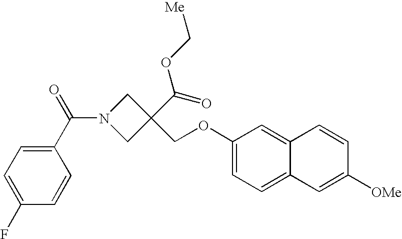

Ethyl 3-{[(6-methoxy-2-naphthyl)oxy]methyl}azetidine-3-carboxylate tosylate (200.0 g, 410 mmol) (Preparation 32) was suspended in ethyl acetate (2800 mL) and then triethylamine (114.3 mL, 820 mmol) was added. The reaction mixture was cooled to 0° C. and 4-fluorobenzoyl chloride was added. The reaction mixture was allowed to stir for 1 hour, after which time 1M citric acid (1000 ml) was added and the layers were separated. The organic layer was washed with 5% w/w aqueous potassium carbonate solution (1000 mL) and twice with water (2×1000 mL). The ethyl acetate was removed under atmospheric pressure and replaced with isopropanol. The precipitate was collected and washed twice with cold isopropanol (2×500 mL). The solid was then dried under reduced pressure to afford the title compound as a white solid in 90% yield, 161.2 g. ¹H NMR (400 MHz, CDCl₃) δ: 1.31 (t, 3H), 3.91 (s, 3H), 4.27-4.47 (m, 7H), 4.74 (m, 1H), 7.10-7.17 (m, 6H), 7.63-7.74 (m, 4H).

1. A compound of formula (I): 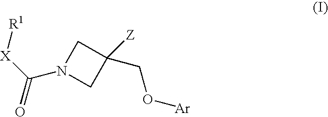
Wherein:
R¹ is a phenyl group, optionally substituted by one or two substituents independently selected from F, Cl, Br, CN, C₁₋₄ alkyl, C₁₋₄ alkylthio and C₁₋₄ alkoxy, per-fluoro-C₁₋₆alkyl and perfluoro-C₁₋₆ alkoxy, or a tetrahydropyranyl group;
X represents a direct link or NH;
Z is selected from 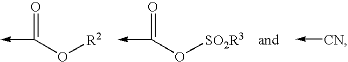
R² and R³, R⁴ and R⁵ are H or C₁₋₆ alkyl, optionally substituted by 1 to 3 fluorine atoms;
Ar is an aromatic group consisting of 1, 2 or 3 aromatic rings, which aromatic rings are independently selected from phenyl and a 5- or 6-membered heteroaromatic ring containing 1, 2 or 3 heteroatoms independently selected from N, O and S; and which aromatic rings, if there are 2 or more, can be fused or linked by one or more covalent bond, and which aromatic rings are optionally substituted by 1, 2 or 3 substituents independently selected from F, Cl, CN, OH, C₁₋₆ alkyl, C₁₋₆ alkylthio, per-fluoro-C₁₋₆ alkyl, perfluoro-C₁₋₆ alkylthio, perfluoro-C₁₋₆ alkoxy, C₁₋₆ alkoxy, SO₂R⁴, NR⁵R⁶, NHSO₂R⁷, SO₂NR⁸R⁹, CONR¹⁰R¹¹, NHCOR¹²;
R⁴, R⁵, R⁶, R⁷, R⁸, R⁹, R¹⁰, R¹¹ and R¹² are H or C₁₋₆ alkyl, optionally substituted by 1 to 3 fluorine atoms; or a pharmaceutically acceptable salt, solvate, hydrate, or prodrug thereof.

2. A compound of claim 1 wherein R¹ is a phenyl group, optionally substituted by one or two substituents independently selected from F, Cl, C₁₋₄ alkyl, C₁₋₄ alkylthio and C₁₋₄ alkoxy, or a tetrahydropyranyl group, or a pharmaceutically acceptable salt, solvate, hydrate, or prodrug thereof.

3. A compound of claim 1 wherein X is a direct link, or a pharmaceutically acceptable salt, solvate, hydrate, or prodrug thereof.

4. A compound of claim 1 wherein Z is 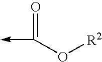
or a pharmaceutically acceptable salt, solvate, hydrate, or prodrug thereof.

5. A compound of claim 1, wherein Ar is a biphenyl, pyridinylphenyl, or naphthyl, optionally substituted by 1, 2 or 3 substituents independently selected from F, Cl, CN, C₁₋₆ alkyl, C₁₋₆ alkylthio, per-fluoro-C₁₋₆ alkyl, perfluoro-C₁₋₆ alkylthio, perfluoro-C₁₋₆ alkoxy, C₁₋₆ alkoxy, SO₂R⁴, NR⁵R⁶, NHSO₂R⁷, SO₂NR⁸R⁹, CONR¹⁰R¹¹, NHCOR¹², or a pharmaceutically acceptable salt, solvate, hydrate, or prodrug thereof.

6. A compound of claim 1, selected from the group consisting of:
1-(4-chlorobenzoyl)-3-{[(4′-fluorobiphenyl-4-yl)oxy]methyl}azetidine-3-carboxylic acid,
1-(4-chlorobenzoyl)-3-{[4-(5-chloropyridin-2-yl)phenoxy]methyl}azetidine-3-carboxylic acid,
3-{[(4′-cyanobiphenyl-4-yl)oxy]methyl}-1-(4-fluorobenzoyl)azetidine-3-carboxylic acid,
3-{[(4′-chlorobiphenyl-4-yl)oxy]methyl}-1-(4-fluorobenzoyl)azetidine-3-carboxylic acid,
1-(4-fluorobenzoyl)-3-{[(6-methoxy-2-naphthyl)oxy]methyl}azetidine-3-carboxylic acid,
1-(4-fluorobenzoyl)-3-{[(4′-fluorobiphenyl-4-yl)oxy]methyl}azetidine-3-carboxylic acid, or a salt, solvate, hydrate, or prodrug thereof.

7. A compound of claim 6, selected from the group consisting of:
1-(4-chlorobenzoyl)-3-{[(4′-fluorobiphenyl-4-yl)oxy]methyl}azetidine-3-carboxylic acid,
1-(4-fluorobenzoyl)-3-{[(6-methoxy-2-naphthyl)oxy]methyl}azetidine-3-carboxylic acid, or a salt, solvate, hydrate, or prodrug thereof.

8. A pharmaceutical composition comprising a compound of claim 1, or a pharmaceutically acceptable salt, solvate, hydrate, or prodrug thereof, and a pharmaceutically acceptable diluent, carrier or adjuvant.

9. A method of treating endometriosis, uterine fibroids, menorrhagia, adenomyosis, primary and/or secondary dysmenorrhoea, or chronic pelvic pain syndrome in a mammal in need of such treatment which comprises administering to said mammal a therapeutically effective amount of a compound of claim 1, or a pharmaceutically acceptable salt, solvate, hydrate, or prodrug thereof.

10. A compound of formula (II), (IV), (V), (VI), (VII), (IX), (XI), (XII) or (XIII) as described herein.
